SEQUENCE LISTING (13 sequences)

SEQ ID NO: 1 - DNA, 10667 bp, Homo sapiens
agcgccccctccgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttca

SEQ ID NO: 2 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 3 - DNA, 10063 bp, Mus musculus
cagcgccccctcggagatccctaggagccagcctgctgggagaaccagagggtccggagcaaacctggaggctgagagggcatcagaggggaaaagactgagctagccactccagtgccatacagaagcttaagggacgcaccacgccagccccagcccagcgacagccaacgcctgttgcagagcggcggcttcgaagccgccgcccaggagctgccctttcctcttcggtgaagtttctaaaagctgcgggagactcagaggaagcaaggaaagtgtccggtaggactacggctgcctttgtcctcttcccctctacccttaccccctcctgggtcccctctccaggagctgactaggcaggctttctggccaaccctctcccctacacccccagctctgccagccagtttgcacagaggtaaactccctttggctgagagtaggggagcttgttgcacattgcaaggaaggcttttgggagcccagagactgaggagcaacagcacgcccaggagagtccctggttccaggttctcgcccctgcacctcctcctgcccgcccctcaccctgtgtgtggtgttagaaatgaaaagatgaaaaggcagctagggtttcagtagtcgaaagcaaaacaaaagctaaaagaaaacaaaaagaaaatagcccagttcttatttgcacctgcttcagtggactttgaatttggaaggcagaggatttccccttttccctcccgtcaaggtttgagcatcttttaatctgttcttcaagtatttagagacaaactgtgtaagtagcagggcagatcctgtcttgcgcgtgccttcctttactggagactttgaggttatctgggcactccccccacccaccccccctcctgcaagttttcttccccggagcttcccgcaggtgggcagctagctgcagatactacatcatcagtcaggagaactcttcagagcaagagacgaggaggcaggataagggaattcggtggaagctacagacaagctcaaggatggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtatcctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaaacccaccttgttccctttccagatgtcttctgcctgttatataactctgcactacttctctgcagtgccttgggggaaattcctctactgatgtacagtctgtcgtgaacaggttcctcagttctatttcctgggcttctccttctttttttttcttcttccctccctctttcaccctcccatggcacattttgaatctgctgcgtattgtggctcctgcctttgttttgatttctgttgtatttctttgaatctgtgatgatcctcttgtggcccagtgtcaattgtgcttgtttatagcactgtgctgtgtgccaaccaagcaaatgtttactcaccttatgccatggcaaatttagagagctataagtatctggagaagaaacaaacagagagaataaaaagcaaaaacaaaaccaaaaaataaaaaaaacacaaacaaaaaacaaaaccaacaaacaaaacatgctaggtttgtttcttcgtggtatacaaataaacacataggattcccaaagaagccgacagtgactagaagaaagtaaaaaattacaaatccacgaggagtcactgtttttgttcatcctgtttctctgtgggaaacttcagttgttgttaatggctattgccattaaagagcaggttgaccccaaagctttactgatagggtagagagaaaagaggacaaggagggcagatggataaccattacctccccacagcctttgtccctgagtcctagagtgctcagttgcagtgtagttccttgtactgaaatgtgcttcttgtttgaaaacttgtctgcatgtgaatgcctcttccttccaatccttttctctcttaacctctgcttccaccctcaattgactttcaatagcttttctcagagctttgtactatatgctctctttagccaaaacttggccactttcactgaagttatgtcagtgagaagaaagtggaaaggtctgactctttggaaggctctattcagatttatgttcatatttccatgtgtgagccatagcggagctttgtgactggagtcagaggaaaaggaagtgatggcttagccattctcccattagagatagtgaatgatgatgccatagtgcaatcatcctttcctctgcttttaaaggacctagagaccccatgcagccacattctccctgcacaagtcttcagtgttcagtggccctgaacttcaccaaaatgcatttaagccaaggtggtaaagcttgtacacttctttggacgtgtttgtagacactgctaagatctccctctcaccaccaccacaaaggctagcaggccagcagccacagcatctatgtttagatgttaatagcataaaagacatctcactcaatgtctttcatcaacagtaaatttctggagcccttagaaaaattggaaagaaagcatcaaagggaccagacaaaatgggcatcttgcccttgtcctccagagacaatatattcctcccaagtggagaaatgtcaatttcctcctcagaacaattaaaggggctacccagaccatggtggaagagaaaactaagtaacccagctgagaaaaatgaagacactagaaccagaaagcacaggactttttcctttccatccagcatacccattggcagaaataatggaaggaaaagagaaggccagaagaaaatacagactgctgaagtcttcagaggcaaagtctaaagccagatgaataccatctggctagatgggcatcagtttgctcatcctcctctattgccattgctgggctgactttggccaaagttacttcgaatctccaccatagttgtcccctctcagtcagagggtgcaggaccactgaaacattctatccaccgtgactctcattggacagatctggccggtgtggctacaaatagactgcacccataaactcagggcaagccctgggtcactggtttcatgtagtctgttgacagccttctttactgtggactctgttcctcaaccttgagtgcaggaggaatgcacatctacttttgcctttgtatcattcctcctcactcagctcttcacctccctgcagaccttaagaaatcaggggccagctgccaagctgactcttttggttggtactatgttaactgaaaaggtgatttccgaaggacaggttttcttccctgatttctttgttgctattaatagcaaaaacaaacttgcaaaacaacttctttaacaaggaagggaggatatatacaatgggtgatatggtaatccaaccctgcttgacaaaaactgaagctgacaggttacatttaaaaacaaaacaaaacaaaacgggacagtttctgatttgctttgtgacaacaccatctggcttatgtacaggagctctcttagctgttccttaaacagaaaaaaaatcattactccttttagttaaatttggttacattttaatagtttctttacatctattctgaagcaatttttgtcttctgtggtacatggattttattataacattctaatatttgtctttgtaaatactagagactctttgatccatttctctaggaagtttttcatcttatggagttctgaatcatgacttttatctttatgaatgtatatgctttttacttgcaaaagccaaaaagagtgaaacagcagtgcaattaaagcaacaccaactaaactccaaatttccaagtgacaatattagagaaaaacagcatacacatggctttatgcctactgcttctgcggtggggtttgggtgcgcaatggaaactgtagcttggctgtgttctcccacacaagtgaagaagagattggtttttgcttttttggattttgtgtttcttttctgttttgttttgttttgttttgttttgctttgctttctttggccatcaatgttccaactaatatgattggcggagcacgtgctctgctcagtagagtgaatgttgctggtgcactatgctcacctgtgaacggctggccatttctccattcatatggttaagatggaagatgaggatcacttaccagagaagtcaaggtgatcatctccaaagaggtttacagtgcttggtaggaatggaaaatgaggacaagaaaaagaggagaaccatggagaaggcccaactgggcaggacagcagccagctgccaaagtcacgaactctgggattcaagaagagtcgtgtagtgctttcaactctcatccgcaggcagctcactgtgtgtggactctgagctgacacgggagttggcttctttgttccatagattttctatgccacaggcaatattattgttcttggaaagttcattatttttttaaattaccttactctcagaaagggatttttttgaaggattctgtcatatatctttggaaaacagaaaatcagtaatatgtatatttttatgtatgttcactggcactaaaaaaaaaaaaaaaaagaaaagaaaaaaaagagaaaaaaaaaagcttcactctgtcctttgggtagttgctgaggttaattgtccaggttgagaaatgtgcttctgctaacatccttctctgtccacactctatttctaagtacatataggcatatataggaagatatattcaacacactttaagaaaaaagtatgtccaccatccacatgataaccacaatgatactccacaaattacatgactttaagcttcaagcaacttctaactgattcattcatttatagccttgccctcttctttcccttaaatttggcccagcacaaagacccaagccacccttatacctccctaagacttaagccagcaccagacttcagaaggttttctgaagacaactgacttgctatccctgcatgaccctagcatggtcctgcaaacacaagagactaattataattctcctccactaattgcctgggtcacaggtcattgggccaaggccatgattcttatgcttacgaaccactaatgctaacctactagattaaatcctgaactgaaagttaaaagaagccatttagcatgtgaaacttcttggagtaagaagtttctgtcccggctgcctttgcaaacaggtttgctttcaccacttatctccttgaaaatctttgaaggccttttttttttaagtagaaaaggagatgaaagcattatattatgtaaccaaagattatattgtatctaagataccaaattttttaagggcagggaaggagcaagcattagtgcctctttggtaaattatccaaagacagactgaaggacttttctgatgattgacttagaagactttgtggggaggggttgtctcacaatatacatatttagaagtgttgagaataatttggggggaaatgggattatagtgtccttcactaactgattttataagcagaactagctttccttttttttttttttaaagtagttacaaagcaaattcttaaagctccatctttgcatggttagaaatggagctggtcttggccactgtgtttactagtgcccatgttagcttatttgaagatgtgaagcccttgataagaaggggtacatttaaaggattagatttttgcactagaaggagggcaggcagaaaccctcatttctgcccagtttggacagcacaaaaagttctctgcagtttaaggcagaaagttgaaatatattgtaaatgagtatttgtatccatgtttcaaaactgaattctatatatagatgtaatgtgttctgatagctttacctttctctgcacctttatatttggttccaggtcatatctgatgccatgtacttgtaagagaggttgcagttacatttttggatgctctctcagaatggataagacacctggattgatcagataactgagatctcttcccttcttgggcctggtgttgaggccttgcaaaggggtggaagaggaaagggtagggtacatgatgtattgcactttactagcttaagacggatgaatgtggaaagggtggtgaaatttcattgaaaatgcctaggaattgcaatagggagaaatccagatgtggggccaggtgcccacccaaaggactggccagcagcctcttcatgggatctgaggcattgggaaaaggaaggctatttccttggttttcaccatccttgttagagaagggcagttgcctggtcttgggaacctggagcaaacgctccttctgtcacatcaattctttcccctgcaattgaggtgctcttgctactgggtgtccgtgtgctctaattctggttctggatatgttctgtaaagattttgataattgctaatgtatttttctctgttaaaaatttgttagtgtgttagaagtcatatctctgtaggtacagatcctttgctacccatgagtagagggattttttttcttcaattaagagtttgaccctggggtctgttgcccagagcccatccagaaaaaaaaatccacatttgtcacaatttttctgaaatttcagtcaaggtaacagatcgctgggagttctctttacccccccaaaaaagcagataattgaatttagcaggtggtgttttagagcaaaaaacaaaacagcctttgacccagctttaatatgacccaatttaatctggccaggaagcaggtaatgtgtattaattggcttccaatcctggttgagtgtagcaaggttctactttgtttcctagttccttttgttacatggcctttcacagaaaggattgactgggtttgcagtatatcttatggccttagcacctattgctaactgtcctgaagggaattgcctatggggttgtcctataagccacttctatcattaaaagcagccaccaatggaatctcccaggtttgaaaaaaaaaaaaaaaaacagatggtcctttaccattcattgacacacatccctgctttcctgtagacagattgactggacattgattagggaatacatggcaaatgacatgcttacactaccctggagattaatttggcagtaggagggaatagacaatgtaaccaagaatgtaatgtaattcttatagagataagaattaaatctggatgtggagagagcaaagagagaaagcattcaatttttttttcaaaagaaaccaatttattttgcttgaaacttctttcgctggggcttcagttctcacagcggctcttggtctccactgggcagcaggaccagccccaagcgctagtgttctgttctctttttgtaatcttggaatcttttgttgctctaaatacaattaaaaatggcagaaacttgtttgttggaatac

SEQ ID NO: 4 - protein, 899 aa, Mus musculus
MEVQLGLGRVYPRPPSKTYRGAFQNLFQSVREAIQNPGPRHPEAANIAPPGACLQQRQETSPRRRRRQQHTEDGSPQAHIRGPTGYLALEEEQQPSQQQAASEGHPESSCLPEPGAATAPGKGLPQQPPAPPDQDDSAAPSTLSLLGPTFPGLSSCSADIKDILNEAGTMQLLQQQQQQQQHQQQHQQHQQQQEVISEGSSARAREATGAPSSSKDSYLGGNSTISDSAKELCKAVSVSMGLGVEALEHLSPGEQLRGDCMYASLLGGPPAVRPTPCAPLPECKGLPLDEGPGKSTEETAEYSSFKGGYAKGLEGESLGCSGSSEAGSSGTLEIPSSLSLYKSGALDEAAAYQNRDYYNFPLALSGPPHPPPPTHPHARIKLENPLDYGSAWAAAAAQCRYGDLGSLHGGSVAGPSTGSPPATTSSSWHTLFTAEEGQLYGPGGGGGSSSPSDAGPVAPYGYTRPPQGLTSQESDYSASEVWYPGGVVNRVPYPSPNCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSNAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 5 - DNA, 4136 bp, Rattus norvegicus
atccctaggagccagcctgctgggagaaccagagggtccggagcaaacctggaggctgagagggcatcagaggggaaaagactgagctagccactccagtgccatacagaagcttaagggacataccacgccagccccagcccagcgacagccaacgcctgttgcagagcggcggcttcgaagccgccgcccagaagctgccctttcctcttcggtgaagtttctaaaagctgcgggagactcggaggaagcgaagaaagtgtccggtaggactacgactgcctttgtcctcctccctcctacccctacccctcctgggtcccctctccctgagcggactaggcaggcttcctggccagccctctcccctacaccaccagctctgccagccagtttgcacagaggtaactccctttggctgaaagcagacgagcttgttgcccattggaagggaggcttttgggagcccagagactgaggagcaacagcacgctggagagtccctgattccaggttctcccccctgcacctcctactgcccgcccctcaccctgtgtgtgcagctagaattgaaaagatgaaaagacagttggggcttcagtagtcgaaagcaaaacaaaagcaaaaagaaaacaaaaagaaaatagcccagttcttatttgcacctgcttcagtggacattgactttggaaggcagagaattttccttccccccagtcaagctttgagcatcttttaatctgttcttcaagtatttagggacaaactgtgaaactagcagggcagatcctgtctagcgcgtgccttcctttacaggagactttgaggctatctgggcgctcccccccccctccctgcaagttttcttccctggagcttcccgcaggtgggcagctagctgcagatactacatcatcagtcagtagaactcttcagagcaagagacgaggaggcaggataagggaattcggtggaagctagagacaagctaaaggatggaggtgcagttagggctgggaagggtctacccacggcccccgtccaagacctatcgaggagcgttccagaatctgttccagagcgtgcgcgaagcgatccagaacccgggccccaggcaccctgaggccgctagcatagcacctcccggtgcctgtttacagcagcggcaggagactagcccccggcggcggcggcggcagcagcaccctgaggatggctctcctcaagcccacatcagaggcaccacaggctacctggccctggaggaggaacagcagccttcacagcagcagtcagcctccgagggccaccctgagagcggctgcctcccggagcctggagctgccacggctcctggcaaggggctgccgcagcagccaccagctcctccagatcaggatgactcagctgccccatccacgttgtccctactgggccccactttcccaggcttaagcagctgctccgcagacattaaagacatcctgagcgaggccggcaccatgcaacttcttcagcagcagcagcaacagcaacagcagcagcagcagcagcagcagcagcagcagcaacagcagcaggaggtaatatccgaaggcagcagcagcgtgagagcaagggaggccactggggctccctcttcctccaaggatagttacctagggggcaattcgaccatatctgacagtgccaaggagttgtgtaaagcagtgtctgtgtccatggggttgggtgtggaagcactggaacatctgagtccaggggagcagcttcggggcgactgcatgtacgcgtcgctcctgggaggtccacccgccgtgcgtcccactccttgtgcgcctctggccgaatgcaaaggtctttccctggacgaaggcccgggcaaaggcactgaagagactgctgagtattcctctttcaagggaggttacgccaaagggttggaaggtgagagtctgggctgctctggcagcagtgaagcaggtagctctgggacacttgagatcccgtcctcactgtctctgtataagtctggagcagtagacgaggcagcagcataccagaatcgcgactactacaactttccgctcgctctgtccgggccgccgcaccccccgccccctacccatccacacgcccgcatcaagctggagaacccgttggactacggcagcgcctgggctgcggcggcagcgcaatgccgctatggggacttggctagcctacatggagggagtgtagccggacccagcactggatcgcccccagccaccgcctcttcttcctggcatactctcttcacagctgaagaaggccaattatatgggccaggaggcgggggcggcagcagtagcccaagcgatgctgggcctgtagccccctatggctacactcggccccctcaggggctggcaagccaggagggtgacttctctgcctctgaagtgtggtatcctggtggagttgtgaacagagtcccctatcccagtcccagttgtgttaaaagtgaaatgggaccttggatggagaactactccggaccttatggggacatgcgtttggacagtaccagggaccacgttttacccatcgactattacttcccaccccagaagacctgcctgatctgtggagatgaagcttctggttgtcactacggagctctcacttgtggcagctgcaaggtcttcttcaaaagagctgcggaagggaaacagaagtatctatgtgccagcagaaatgattgcaccattgataaatttcggaggaaaaattgtccatcgtgtcgtctccggaaatgttatgaagcagggatgactctgggagctcgtaagctgaagaaacttggaaatctcaaactacaggaagaaggagaaaactccagtgctggtagccccactgaggacccatcccagaagatgactgtatcacacattgaaggctatgaatgtcaacctatctttcttaatgtcctggaagccattgagccaggagtggtgtgtgccggacatgacaacaaccagcctgattcctttgctgccttgttatctagtctcaacgagcttggcgagagacagcttgtacatgtggtcaagtgggccaaggccttgcctggcttccgcaacttgcatgtggatgaccagatggcagtcattcagtattcctggatgggactgatggtatttgccatgggttggcggtccttcactaatgtcaactctaggatgctctactttgcacctgacctggttttcaatgagtatcgcatgcacaagtctcgaatgtacagccagtgcgtgaggatgaggcacctttctcaagagtttggatggctccagataaccccccaggaattcctgtgcatgaaagcactgctactcttcagcattattccagtggatgggctgaaaaatcaaaaattctttgatgaacttcgaatgaactacatcaaggaacttgatcgcatcattgcatgcaaaagaaaaaatcccacatcctgctcaaggcgcttctaccagctcaccaagctcctggattctgtgcagcctattgcaagagagctgcatcaattcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaacccaccttgttcccttttcagatgtcttctgcctgttatataactctgcactacttctctgcagtgccttgggggaaattcctctactgatgtacagtctgtcatgaacatgttccccagttctatttcctgggcttttccttctttctttttcttcttctctgcctctcttaccctcccatggcacattttgaatccgctgcgtgttgtggctcctgcctgtgttttgagttttgttgtatttcttcaagtctgtgatgatcttcttgtggcccagtgtcaactgtgcttgtttatagcactgtgctgtgtgccaaccaagcaaatgtttactcaccttatgccatggcaagtttagagagctataagtatcttgggaagaaacaaacagagagagtaaaaaaacc

SEQ ID NO: 6 - protein, 902 aa, Rattus norvegicus
MEVQLGLGRVYPRPPSKTYRGAFQNLFQSVREAIQNPGPRHPEAASIAPPGACLQQRQETSPRRRRRQQHPEDGSPQAHIRGTTGYLALEEEQQPSQQQSASEGHPESGCLPEPGAATAPGKGLPQQPPAPPDQDDSAAPSTLSLLGPTFPGLSSCSADIKDILSEAGTMQLLQQQQQQQQQQQQQQQQQQQQQQEVISEGSSSVRAREATGAPSSSKDSYLGGNSTISDSAKELCKAVSVSMGLGVEALEHLSPGEQLRGDCMYASLLGGPPAVRPTPCAPLAECKGLSLDEGPGKGTEETAEYSSFKGGYAKGLEGESLGCSGSSEAGSSGTLEIPSSLSLYKSGAVDEAAAYQNRDYYNFPLALSGPPHPPPPTHPHARIKLENPLDYGSAWAAAAAQCRYGDLASLHGGSVAGPSTGSPPATASSSWHTLFTAEEGQLYGPGGGGGSSSPSDAGPVAPYGYTRPPQGLASQEGDFSASEVWYPGGVVNRVPYPSPSCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSSAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 7 - protein, 8 aa, Artificial Sequence
  Synthetic peptide
SIINFEKL

SEQ ID NO: 8 - protein, 9 aa, Artificial Sequence
  Synthetic peptide
RLQGISPKI

SEQ ID NO: 9 - protein, 9 aa, Artificial Sequence
  Synthetic peptide
LLLFSIIPV

SEQ ID NO: 10 - protein, 10 aa, Artificial Sequence
  Synthetic peptide
RMLYFAPDLV

SEQ ID NO: 11 - protein, 9 aa, Artificial Sequence
  Synthetic peptide
FLCMKALLL

SEQ ID NO: 12 - protein, 9 aa, Artificial Sequence
  Synthetic peptide
QLTKLLDSV

SEQ ID NO: 13 - protein, 9 aa, Artificial Sequence
  Synthetic peptide
KASEKIFYV

(19) United States
(12) Patent Application Publication
(10) Pub. No.: US 2022/0347282 A1
(43) Pub. Date: Nov. 3, 2022

(54) ANTI-TUMOR DNA VACCINE WITH PD-1 AND LAG-3 PATHWAY BLOCKADE

(71) Applicant: WISCONSIN ALUMNI RESEARCH FOUNDATION, Madison, WI (US)

(72) Inventors: Douglas McNeel, Madison, WI (US); Christopher D. Zahm, Madison, WI (US); Jena Moseman, Madison, WI (US)

(21) Appl. No.: 17/732,237

(22) Filed: Apr. 28, 2022

Related U.S. Application Data
(60) Provisional application No. 63/180,726, filed on Apr. 28, 2021.

(51) Int. Cl. A61K 39/00; A61P 35/00; A61K 39/395; A61P 37/04

(52) U.S. Cl. CPC A61K 39/001184; A61P 35/00; A61K 39/001102; A61K 39/001194; A61K 39/001106; A61K 39/001193; A61K 39/3955; A61P 37/04; A61K 2039/53; A61K 2039/884; A61K 2039/868; A61K 2039/892; A61K 2039/505; A61K 2039/572; A61K 2039/545

(57) ABSTRACT

The present invention provides combination therapies and methods of treating cancer, including, cancers that are resistant to PD-1 therapy. The combination therapies described herein comprise a DNA vaccine to a tumor antigen, anti-PD-1 therapy, and an anti-LAG-3 therapy, which provides an increased T cell response against the cancer.

### CROSS-REFERENCE TO RELATED APPLICATIONS

This application claims the benefit of priority under 35 U.S.C. § 119(e) of U.S. Provisional Application No. 63/180,726, filed Apr. 28, 2021, the contents of which is incorporated herein by reference in its entirety.

### STATEMENT REGARDING FEDERALLY SPONSORED RESEARCH

This invention was made with government support under CA219154 awarded by the National Institutes of Health. The government has certain rights in the invention.

### SEQUENCE LISTING

A Sequence Listing accompanies this application and is submitted as an ASCII text file of the sequence listing named “960296_04284_ST25.txt” which is 57,623 bytes in size and was created on Apr. 28, 2022. The sequence listing is electronically submitted via EFS-Web with the application and is incorporated herein by reference in its entirety.

### BACKGROUND

The blockade of T-cell immune checkpoint receptors to enable the activity of tumor-specific T cells has revolutionized the treatment of cancer. Notably, an antibody blocking cytotoxic T-lymphocyte-associated protein 4 (CTLA-4) was the first of these agents that was FDA approved in 2010 for the treatment of metastatic melanoma¹. Other immune checkpoint receptors were discovered as markers of cell death and exhausted, non-functional T cells that had experienced long-term antigen exposure². In particular, the programmed death 1 (PD-1) receptor, while initially thought to indicate T cell exhaustion, was subsequently found to function by preventing functional Th1 CD8+ T cells from causing autoimmunity³. The immunosuppressive activity of PD-1 is executed following ligand (PD-L1) encounter on self (including tumor) cells resulting in the activation of a signaling pathway that attenuates cytotoxic T cell activity³⁻⁵. As a result, remarkable anti-tumor activity can be achieved by blocking PD-1/PD-L1 ligation using antibodies, and this approach has led to multiple new FDA approvals over the last 5 years, underscoring the power of this single immune checkpoint⁶⁻⁸.

The general rationale for use of T-cell checkpoint blockade as cancer therapies is that ligand-induced checkpoint signaling leads to the activation of regulatory pathways within tumor-reactive T cells and thus blocking ligand interaction can remove the negative signal to allow for eradication of tumor cells. As PD-1 and other checkpoints operate through distinct mechanisms but result in similar outcomes, it follows that simultaneous blockade could have a synergistic effect. Indeed, a number of murine and clinical studies have been conducted using PD-1 blockade with other checkpoint blocking therapies⁹⁻¹³. Preclinical studies demonstrate that blocking checkpoints with complementary mechanisms of action can result in the expansion of unique T-cell repertoires and activate adaptive anti-tumor immunity¹⁰. Furthermore, a randomized, double-blind, phase 3 study of PD-1 blockade alone or in a dual blockade combination with CTLA-4 blockade in patients with metastatic melanoma found a median progression-free survival of 11.5 months with the combination and 6.9 months with single agent PD-1 blockade⁹. Similar results in patients with renal cell cancer have led to the FDA approval of CTLA-4 and PD-1 dual blockade for the treatment of metastatic renal cell cancer and melanoma^{14,15}.

In previous studies, the inventors found that DNA or peptide vaccine-induced activation of tumor-specific, CD8+ T cells led to increased expression of multiple checkpoint receptors that could mitigate the anti-tumor response following vaccination. More specifically, the inventors found that antigens with high-affinity for MHC-I increased contact time between CD8+ T cells and APCs, which led to increases in multiple immune checkpoints, including PD-1, CTLA-4, lymphocyte activation gene-3 (LAG-3), and T-cell immunoglobulin and mucin-domain containing-3 (TIM-3) on responding cells when compared to cells activated with lower affinity epitopes. Additionally, the reduced anti-tumor efficacy could be recovered when vaccines encoding high-affinity epitopes were combined with PD-1 or PD-L1 blocking antibodies^{16,17}. In a separate study, the inventors found that immunization approaches leading to increased antigen expression also led to increased LAG-3 on tumor antigen-specific CD8+ T cells, which was likewise capable of interfering with the anti-tumor response. Again, the reduced anti-tumor efficacy could be recovered when vaccination was combined with LAG-3 blocking antibodies¹⁸. These data demonstrate that blocking the regulatory pathways induced with vaccination can enhance anti-tumor responses and indicate checkpoint receptor upregulation as a major mechanism of tumor resistance to vaccination. Furthermore, these data demonstrate that T-cell activating therapies can result in the expression of multiple, different checkpoint receptors, and hence combination blockade might be preferable. This is particularly relevant for anti-tumor DNA vaccines, which result in tumor-antigen presentation via professional APC and/or bystander cells. Presentation by multiple cell types may increase the diversity of responding T cells and likewise the complexity of checkpoint expression profiles on these populations^{18,19}. Consequently, the inventors hypothesized that blockade of multiple checkpoints may be necessary to elicit CD8+ T cells with greater anti-tumor activity in the context of anti-tumor immunization.

### SUMMARY OF INVENTION

The present invention is a method of enhancing DNA vaccine activity through the addition of inhibitors to the PD-1 and LAG-3 checkpoint pathways. The inventors have surprisingly discovered an enhancement of PD-1 expression and LAG-3 in T cells after treatment with a DNA vaccine. Using a combination of inhibitors for PD-1 and LAG-3 (antibodies against PD-1 and LAG-3) with a DNA vaccine resulted in an increase in T cell activity, which is not seen with other combinations of inhibitors, such as using inhibitors of the CTLA-4 or TIM-3 pathways. The blockade of PD-1 and LAG-3 pathways caused a more robust immune response to the inventors' DNA vaccine in a prostate cancer mouse model.

In one aspect, the disclosure provides a method of treating a subject having cancer, the method comprising administering an anti-tumor vaccine and a combination of a PD-1 inhibitor and an LAG-3 inhibitor, wherein the combination is effective in increasing the efficacy of the anti-tumor vaccine and treating the cancer.

In another aspect, the disclosure provides a method of increasing the anti-tumor T cell response to a tumor antigen in a subject having cancer, the method comprising administering an effective amount of a DNA vaccine and a combination of PD-1 inhibitor and an LAG-3 inhibitor, wherein the combination is effective in increasing the anti-tumor T cell immune response.

In a further embodiment, the disclosure provides a method of increasing the immune response to a tumor antigen on a cell in a subject, the method comprising contacting the subject with at least one vaccine directed to said tumor antigen, at least one PD-1 inhibitor and at least one LAG-3 inhibitor, wherein the immune response to said tumor antigen is increased relative to a subject treated with the tumor vaccine alone.

In yet another embodiment, the disclosure provides a kit for eliciting an anti-tumor response, the kit comprising: at least one DNA vaccine to a tumor antigen; at least one PD-1 inhibitor; and at least one LAG-3 inhibitor.

### BRIEF DESCRIPTION OF THE DRAWINGS

FIG. 1. T-cell activation by professional APCs can lead to distinct immune checkpoint expression on CD8+T cells. Splenocytes were prepared from the spleens of OT-1 mice and separated into T cells (CD8+) and B-cells (CD19+) using MACS. DC (CD11c+) were prepared from the spleens of Flt3 ligand-treated B6 mice. T cells were stimulated with a control peptide (No Stim), the SIINFEKL peptide alone (No APC), or the SIINFEKL peptide in combination with either B cells or DC. After 72 hours the cells were collected and the checkpoint and 4-1BB expression analyzed by flow cytometry. Shown is the mean fluorescence intensity (MFI) and standard error of the mean of 4-1BB, PD-1, CTLA-4, TIM-3, or LAG-3 on CD8+T cells from triplicate assessments (panel A), and a representative histogram for each marker (panel B). Asterisks indicate p<0.05 by one-way ANOVA with Bonferroni's multiple comparisons correction. Results are from one experiment (N=3 mice per group) and are representative of two similar, independent experiments.

FIG. 2. Blockade of PD-1 or LAG-3 improves anti-tumor activity of activated CD8+T-cells. As shown in panel A, B6 mice were inoculated with 1×10⁶ PD-L1-expressing E.G7-OVA cells. After ten days, 1×10⁶ OT-1 T cells, stimulated with or without peptide and with or without DC as in FIG. 1, were adoptively transferred into the tumor-bearing mice. The following day, mice were treated with IgG isotype control (gray), PD-1 blocking (red), LAG-3 blocking (purple), or a combination of both PD-1 and LAG-3 blocking antibodies (green). Tumor growth was measured as indicated on the X axes. Shown in panel B are the growth curves for mice that received T cells which had not been incubated with DC and a nonspecific peptide (No Stim), T without DC cells stimulated with SIINFEKL peptide alone (No APC), or T cells stimulated with peptide in the presence of DC (DC). Panel C shows the same data grouped by checkpoint blockade treatment rather than T-cell stimulation conditions. Measurements for individual mice are shown in Supplemental FIG. 10. Asterisks indicate p<0.05 as assessed by 2-way ANOVA with Bonferroni's multiple comparisons test. Results are from one experiment with N=6 mice per group.

FIG. 3. DNA vaccination can elicit CD8+T cells differentially expressing PD-1 and LAG-3. Panel A: six-week-old HHDII HLA-A2+ mice were immunized with pTVG4 empty vector, the native pTVG-SSX2 DNA vaccine (SSX2), pTVG-SSX2^{HA} (SSX2^{HA}), or MIP-SSX2. Mice were euthanized at the time points indicated and splenocytes were assessed by flow cytometry gated on CD3+ CD8+ tetramer+ cells (panels B and D, n=6 mice/time/condition) or following stimulation with an HLA-A2-restricted peptide epitope (SSX2 p103-111) to determine the number of responding cells via intracellular cytokine analysis (panel C, n=3 mice/timepoint). In panel C, comparisons are of total cytokine-secreting CD8+T cells at each time point between vaccine-treated or pTVG4 control-treated animals. For all panels, asterisks indicate p<0.05 by two-way ANOVA with Bonferroni's multiple comparisons correction. MFI=mean fluorescence intensity. Results are from one experiment and are representative of two similar, independent experiments.

FIG. 4. PD-1 blockade is superior to LAG-3 blockade when used in combination with an anti-tumor DNA vaccine in an αPD-1 sensitive tumor. Panel A: six-week-old HHDII (HLA-A2+) mice were inoculated s.c. with SSX2+ HLA-A2+ sarcoma cells and immunized with pTVG4 empty vector, pTVG-SSX2 (SSX2), pTVG-SSX2^{HA} (SSX2^{HA}), or MIP-SSX2 in combination with αPD-1, αLAG-3, both αPD-1/αLAG-3, or IgG control. Tumor growth was measured over time. Panel B: shown are the tumor growth curves for each vaccine group. Animals with tumors greater than 2 cm³ in size were euthanized, and data were censored at 2 cm³. Panel C: data are presented as survival plots using the time to death or when tumors reached 2 cm³ in size, whichever occurred first. Individual tumor measurements are shown in Supplemental FIG. 13. Asterisks in panel B indicate p<0.05 as assessed by mixed-effects model with Geisser-Greenhouse correction and Tukey's multiple comparisons test with individual variances; N=6 mice/time point/condition. n. s.=not significant. Results are from one experiment (N=6) and are representative of two similar, independent experiments. For data points above the Y axis, statistical comparisons are indicated on the figure legends. In panel C, asterisks indicate p<0.05 as assessed by log-rank test.

FIG. 5. Vaccination with PD-1 and LAG-3 blockade is superior to vaccination with either blockade alone in αPD-1 resistant prostate cancer model. As shown in panel A, six-week-old FVB mice (n=20 per group) were inoculated s.c. with 10⁶ MyC-CaP cells and immunized with pTVG4 empty vector or pTVG-AR in combination with IgG control, αPD-1, αLAG-3, or both αPD-1/αLAG-3 antibodies. Tumor growth was measured over time. Panel B: Shown are the mean tumor growth curves and standard deviations; individual tumor measurements are shown in Supplemental FIG. 15. Animals with tumors greater than 2 cm³ in size were euthanized, and data were censored at 2 cm³. Panel C: data are presented as survival plots using the time to death or when tumors reached 2 cm³ in size, whichever occurred first. Results shown are from one experiment and representative of three independent experiments. Panel D: Shown are the number of CD8+ (top) and CD4 (bottom) tumor-infiltrating lymphocytes per gram of tumor tissue collected at day 29 as determined by flow cytometry (gating strategy shown in Supplemental FIG. 11). Panel E: Shown are the distribution of effector memory (T_{EM}, CD44+ CD62L^{lo}), resident memory (T_{RM}, CD69+ CD103+), central memory (T_{CM}, CD44+ CD62L+), and naïve (T_{NV}, CD44−CD62L+) cells among the CD8+T cells. Asterisks indicate p<0.05 assessed by the mixed-effects model with Geisser-Greenhouse correction and Tukey's multiple comparisons test with individual variances (panel B), by log-rank test (panel C), or by the one-way ANOVA with Tukey's multiple comparisons test (panels D and E).

FIG. 6. Co-culture experimental methods schematic. Shown in A-C are flow diagrams for the isolation/purification of DCs (A), T cells (B) and B cells (C). Panel D indicates which cells were cultured together for the studies described in relation to FIG. 1.

FIG. 7. Immunization studies in HHD-II mice. Shown is a schematic flow diagram of the experiments conducted using SSX2-targeted DNA vaccines in HHD-II mice.

FIG. 8. Tumor treatment studies: E.G7-OVA tumors in B6 mice. Shown is a schematic flow diagram for the studies using adoptive transfer of T cells to E.G7-OVA tumor-bearing mice.

FIG. 9. Tumor treatment studies: SSX2+ sarcomas in HHD-II mice. Shown is a schematic flow diagram of the experiments conducted using SSX2+ sarcomas in HHD-II mice.

FIG. 10. Tumor treatment studies: MycCaP tumors in FVB mice. Shown is a schematic flow diagram of the experiments conducted using DNA vaccines with T-cell checkpoint blockade in MycCaP tumor-bearing FVB mice.

FIG. 11. Gating strategy for tumor-infiltration T cells (TIL) analysis. Shown is the flow cytometry gating strategy employed to assess TIL and memory phenotypes. From left to right, top to bottom, all cells were evaluated by forward and side scatter to include lymphocytes, gated for live events, then gated for CD45 expression, then CD3+CD8+ cells were gated for the expression of memory markers as shown.

FIG. 12. Individual growth curves for FIG. 2, OVA-expressing tumor study. B6 mice were inoculated with 1×106 PD-L1-expressing E.G7-OVA cells. After ten days, 1×106 OT-1 T cells, stimulated with or without peptide and with or without APC as in FIG. 1, were adoptively transferred into the tumor-bearing mice. The following day, mice were treated with IgG isotype control (top row), PD-1 blocking (second row), LAG-3 blocking (third row), or a combination of both PD-1 and LAG-3 blocking antibodies (fourth row). Tumor growth was measured as indicated on the X axes. Shown are the individual tumor measurements for each mouse per day following tumor implantation, and the median tumor size. Results are from one experiment with N=6 mice per group.

FIG. 13. Representative histograms and dot plots for FIG. 3. Six-week-old HHDII HLA-A2+ mice were immunized with pTVG4 empty vector, the native pTVG-SSX2 DNA vaccine, pTVG-SSX2HA, or MIP-SSX2. Splenocytes obtained from mice at different time points were assessed by flow cytometry for expression of various markers, as described in FIG. 3. Shown are representative data for expression of 4-1BB, LAG-3, and PD-1 expression four days following treatment with the different vaccines (panel A). Panel B shows representative dot plots for the intracellular cytokine analysis. The upper left plot shows the gating of live, lymphocyte scatter for CD3 (X axis) by CD8 (Y axis). The indicated gate was used to evaluate expression of the individual cytokines as shown in the other plots. Panel C shows the evaluation for CD44 and CD62L expression gated on live/CD3+/CD8+/tetramer+ cells. Quadrants are based on FMO gating performed at each time point.

FIG. 14. Individual growth curves for FIG. 4, SSX2/HLA-A2 tumor study. Six week-old HHDII (HLA-A2+) mice were inoculated s.c. with SSX2+ HLA-A2+ sarcoma cells and immunized with pTVG4 empty vector (top row), pTVG-SSX2 (second row), pTVG-SSX2HA (third row), or MIP-SSX2 (fourth row) in combination with IgG control (first column), αPD-1 (second column), αLAG-3 (third column), or both αPD-1/αLAG-3 (fourth column), as described in FIG. 4. Tumor growth was measured over time. Shown are the individual tumor measurements for each animal per group and the median (line) for each treatment.

FIG. 15. Vaccination with PD-1 and LAG-3 blockade is superior to antibody treatment alone in MycCaP prostate tumors. Six-week-old FVB mice (n=10 per group) were inoculated s.c. with 106 MyC-CaP cells and immunized with pTVG4 empty vector or pTVG-AR in combination with IgG control or αPD-1 and αLAG-3 antibodies. Tumor growth was measured over time (panel A). Animals with tumors greater than 2 cm3 in size were euthanized, and data are censored at 2 cm3. Kaplan-Meier curves depicting either the time of death or when the tumor reached 2 cm3 in size, whichever occurred first (panel B). Panel C: In a separate study, six-week-old FVB mice (n=5 per group) were inoculated s.c. with 106 MyC-CaP cells and immunized the following day and weekly with pTVG4 empty vector or pTVG-AR. Groups received IgG control, αPD-1, αLAG-3, or both αPD-1 and αLAG-3 antibodies the day after each immunization. On day 29, tumors were collected, digested with collagenase, and evaluated for the presence of CD11b+Gr−1+ (MDSC) cells or CD4+FoxP3+ (Treg) among live cells. These are expressed as an absolute number per gram of tumor. Panel D: Individual growth curves for mice from FIG. 5A. Asterisks in panels A and C indicate p<0.05 assessed by the mixed-effects model with Geisser-Greenhouse correction or two-way ANOVA, both with Tukey's multiple comparisons test with individual variances.

### DETAILED DESCRIPTION

The present invention provides compositions, kits and methods for increasing an immune response to tumor antigens, resulting in the ability to treat or reduce tumor burden in a subject having cancer. The present inventors have found that combinations of PD-1 and LAG-3 blockade in the context of anti-tumor vaccination enhanced vaccine induced anti-tumor responses with all the vaccines tested, especially in cancers that are resistant to PD-1 therapy. In the prostate cancer model, which is resistant to single-agent PD-1 blockade using a vaccine encoding a naturally expressed tumor antigen, the dual blockade group demonstrated greater therapeutic efficacy than other treatment groups. These results indicate that depending on which cells are presenting antigen, tumor-reactive CD8+ T cells can activate with distinct patterns of checkpoint receptor expression and dual blockade of PD-1 and LAG-3 can provide significant benefit over either blockade alone in PD-1 resistant MycCaP prostate tumors.

The inventors' method is based on the finding that T-cell activation following vaccination resulted in the expression of PD-1, LAG-3, CTLA-4, and TIM-3 checkpoint receptors. However, in the absence of professional APC, activated CD8+ T cells expressed only LAG-3. The inventors found that the combination checkpoint blockade following vaccination including LAG-3 blockade can result in antigen presentation through non-professional APC. Not to be bound by any theory, but the inventors believe there are two negative feedback loops at play in the anti-tumor T cell response, the first in which excess TCR stimulation leads to the expression of PD-1 and other inhibitory receptors and molecules; and the second negative feedback loop that is regulated independently of PD-1 and involves LAG-3 expression, and consequently that the use of PD-1 and LAG-3 in a dual checkpoint blockade strategy provides advantages following vaccination with a tumor antigen.

The data collected by the inventors demonstrates that following vaccination in a subject having a PD-1 therapy resistant tumor, there is a benefit of treating with both an anti-PD-1 inhibitor and a LAG-3 inhibitor.

In one embodiment, the present disclosure provides a method of treating a subject having cancer, preferably a cancer that is resistant to anti-PD-1 therapy. The method comprises administering an effective amount of an anti-tumor vaccine and a combination of a PD-1 inhibitor and a LAG-3 inhibitor, wherein the combination of the PD-1 inhibitor and LAG-3 inhibitor is capable of increasing the immune response to the anti-tumor vaccine and in treating the cancer.

The anti-tumor vaccine is a composition comprising a tumor antigen or a polynucleotide encoding the tumor antigen. In one embodiment, the anti-tumor vaccine is a DNA vaccine.

The term “tumor antigen” or “cancer antigen” refers to a protein that is specifically found on tumor cells or may be a molecule that is greatly upregulated on tumor cells. The term antigen refers to the ability of the protein to elicit an immune response when presented by antigen presenting cells to T cells. Suitable tumor antigens are known in the art, and will vary depending on the type of tumor being treated.

In some embodiments, the tumor antigen is synovial sarcoma X breakpoint 2 (SSX2), androgen receptor ligand-binding domain (AR LBD), prostate-specific antigen (PSA), human epidermal growth factor receptor 2 (HER-2/neu), or prostatic acid phosphatase (PAP). In some embodiments, the antigen is a fragment or epitope of the antigen protein.

In some embodiments, the anti-tumor vaccine is a DNA vaccine. Any DNA vaccine that targets cancer may be used with the present methods. DNA vaccines against prostate cancer, breast cancer, ovarian cancer, sarcoma, lymphoma, among others, are known and understood in the art.

In some embodiments, the anti-tumor vaccine is a DNA vaccine comprises a polynucleotide encoding the tumor antigen. For example, suitable tumor antigens include, but are not limited to, synovial sarcoma X breakpoint 2 (SSX2), androgen receptor ligand-binding domain (AR LBD), prostate-specific antigen (PSA), human epidermal growth factor receptor 2 (HER-2/neu), and prostatic acid phosphatase (PAP).

Suitable prostate cancer vaccines for use in the present methods include, for example, recombinant DNA vaccines that encode an androgen receptor or fragments thereof. Suitable recombinant DNA vaccines are disclosed in U.S. Pat. Nos. 7,910,565, 8,513,210 and 8,962,590, each of which is incorporated herein by reference in its entirety. In some embodiments, the DNA vaccine comprises the pTVG-AR vector, which encodes the ligand-binding domain of the human androgen receptor gene inserted into the pTVG4 vector to create the immunization vector pTVG-AR, as disclosed in U.S. Pat. No. 7,910,565. Androgen receptor genes are known and have been cloned from many species. For example, the human, mouse, rat, dog, chimpanzee, macaque, and lemur androgen receptor mRNA that correspond to cDNA along with amino acid sequences can be found at GenBank Accession Nos. NM_000044 (cDNA-SEQ ID NO:1 and amino acid sequence-SEQ ID NO:2), NM_013476 (cDNA-SEQ ID NO:3 and amino acid sequence-SEQ ID NO:4), NM_012502 (cDNA-SEQ ID NO:5 and amino acid sequence-SEQ ID NO:6), NM_001003053, NM_001009012, U94179, and U94178, respectively. According to another embodiment, the DNA vaccine comprises a polynucleotide operatively linked to a transcriptional regulatory element (e.g., a promoter such as a heterologous promoter) wherein the polynucleotide encodes a member selected from (i) a mammalian androgen receptor (e.g., a human androgen receptor), (ii) a fragment of the androgen receptor that comprises the ligand-binding domain, (iii) a fragment of the ligand-binding domain defined by SEQ ID NO:9 (LLLFSIIPV, amino acids 811-819 of SEQ ID NO:2); (iv) a fragment of the ligand-binding domain defined by SEQ ID NO:10 (RMLYFAPDLV, amino acids 761-770 of SEQ ID NO:2), (v) a fragment of the ligand-binding domain defined by SEQ ID NO:11 (FLCMKALLL, amino acids 805-813 of SEQ ID NO:2), and (vi) a fragment of the ligand-binding domain defined by SEQ ID NO:12 (QLTKLLDSV, amino acids 859-867 of SEQ ID NO:2), wherein administration of said vaccine to a subject induces a cytotoxic immune reaction against cells expressing androgen receptor.

Other suitable DNA vaccines encode native or modified SSX2 peptides, as described in Smith et al. 2011 (Vaccines targeting the cancer-testis antigen SSX-2 elicit HLA-A2 epitopes specific cytolytic T cells. J. Immunother. 2011:34:569-80) and Smith et al. 2014 (DNA vaccines encoding altered peptide ligands for SSX2 enhance epitope-specific CD8+ T cell immune responses. Vaccine 2014:32:1707-15), each of which is incorporated herein by reference in its entirety. In some embodiments, the DNA vaccines encoding native or modified SSX2 peptides comprise pTVG-SSX2^{HA} (KASEKIFYV (SEQ ID NO: 13) and/or RLQGISPKI (SEQ ID NO: 8)), MIP-SSX2, details for which can be found in Colluru V T, Zahm C D, McNeel D G. Mini-intronic plasmid vaccination elicits tolerant LAG3(+) CD8(+) T cells and inferior antitumor responses. Oncoimmunology. 2016; 5(10):e1223002, which is incorporated by reference herein in its entirety. Other suitable prostate cancer vaccines include vaccines then encode prostatic acid phosphatase (PAP), for example, those described in U.S. Pat. No. 7,179,797, U.S. application Ser. Nos. 11/615,778, and 15/430,012, each of which is incorporated herein by reference in its entirety.

Suitable dosages and schedules for administering the DNA vaccine would be readily understood by one skilled in the art. An appropriate dosage would depend upon several factors, including the patient (age, weight, etc.), the DNA vaccine in use, the route of administration, and the additional drugs administered in combination with the vaccine. For example, the DNA vaccine may be administered at about 10 μg to −1 mg per dose (e.g., 100 μg). The vaccine may be administered using a standard schedule over a period of months or years.

The methods further comprise a combination of a LAG-3 inhibitor and a PD-1 inhibitor. Lymphocyte activation gene-3 (LAG-3; CD223) is a type I transmembrane protein expressed on the cell surface of activated CD4⁺ and CD8⁺ T cells and subsets of NK and dendritic cells (Triebel F, et al., J. Exp. Med. 1990; 171:1393-1405; Workman C J, et al., J. Immunol. 2009; 182(4): 1885-91). LAG-3 negatively regulates T cell signaling and functions. Suitable LAG-3 checkpoint inhibitors include, but are not limited to, for example, anti-LAG-3 antibody. Anti-LAG-3 antibodies are known in the art and commercially available, for example, relatlimab (Bristol-Myers Squibb). Suitable antibodies are also described in U.S. Pat. No. 9,908,936, the contents of which are incorporated by reference in their entirety. Other suitable LAG-3 checkpoint inhibitors include molecules that can prevent binding of LAG-3 to its ligands (e.g. major histocompatibility class II (MEW II) and/or Galectin-3) or molecules that inhibit signaling through the LAG-3 pathway. Not to be bound by any theory, but the inventors hypothesize that the combination of LAG-3 blockade with PD-1 inhibitor works better than the combination of PD-1 with other checkpoint inhibitors because of the expression levels of PD-1 and LAG3 on CD8+ T cells, making the combination blockade more effective in eliciting an anti-tumor CD8+ T cell response.

The term “antibody” as used herein also includes an “antigen-binding portion” of an antibody. The term “antigen-binding portion,” as used herein, refers to one or more fragments of an antibody that retain the ability to specifically bind to an antigen (e.g., polypeptide or fragment thereof of LAG-3) and block signaling through the LAG-3 pathway. Examples of binding fragments encompassed within the term “antigen-binding portion” of an antibody include, but are not limited to, (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al, (1989) Nature 341:544-546), which consists of a VH domain; and (vi) an isolated complementarity determining region (CDR).

Antibodies used in the methods may be polyclonal or monoclonal; xenogeneic, allogeneic, or syngeneic; or modified forms thereof (e.g., humanized, chimeric, etc.). Suitable antibodies may be fully human or humanized. Preferably, antibodies of the invention bind specifically or substantially specifically to the antigen (e.g. LAG-3, polypeptides or fragments thereof). The term “monoclonal antibodies” refers to a population of antibody polypeptides that contain only one species of an antigen binding site capable of binding a particular epitope of an antigen, whereas the term “polyclonal antibodies” refers to a population of antibody polypeptides that contain multiple species of antigen binding sites capable of interacting with a particular antigen.

Suitable antibodies are able to inhibit or reduce at least one biological activity of the antigen (e.g. LAG-3) it binds. In certain embodiments, the antibodies or fragments thereof substantially or completely inhibit a given biological activity of the antigen.

For the present invention, inhibitors of PD-1 or PD-L1 are contemplated to be used in combination with the LAG-3 inhibitors described herein. PD-1 or PD-L1 inhibitors may include, but are not limited to, antibodies, peptides, small molecules, antisense RNAs, cDNAs, miRNAs, siRNAs, aptamers, oligonucleotides, and the like. Examples include, but are not limited to, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, and anti-PD-1 antibody, available from Merck and Co and described in U.S. Pat. Nos. 8,952,136, 83545509, 8,900,587 and EP2170959; atezolizumab is an anti-PD-L1 available from Genentech, Inc. (Roche) and described in U.S. Pat. No. 8,217,149; avelumab (Bavencio, Pfizer, formulation described in PCT Publ. WO2017097407), durvalumab (Imfinzi, Medimmune/AstraZeneca, WO2011066389), cemiplimab (Libtayo, Regeneron Pharmaceuticals Inc., Sanofi), spartalizumab (PDR001, Novartis), camrelizumav (AiRuiKa, Hengrui Medicine Co.), sintillimab (Tyvyt, Innovent Biologics/Eli Lilly), KN035 (Envafolimab, Tracon Pharmaceuticals); tislelizumab available from BeiGene and described in U.S. Pat. No. 8,735,553; among others and the like. Other PD-1 and PD-L1 that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others. The term “checkpoint inhibitor therapy” refers to the form of cancer immunotherapy that block inhibitory checkpoints and thereby restore immune system function. Such therapies are known by those skilled in the art. In some embodiments, the PD-1 inhibitor is selected from the group consisting of Nivolumab (anti-PD-1), Pembrolizumab (anti-PD-1), and combinations thereof. In some embodiments, the PD-L1 inhibitor is selected from atezolizumab, avelumab, and durvalumab, among others. CTLA-4 inhibitors are not contemplated for use in the present invention, as described in the examples, CTLA-4 inhibitors do not act through the same pathway as the PD-1/PD-L1 inhibitors with respect to NLRP3 inhibitors, and as such, the combination of such does not produce the desired outcome as described herein, demonstrating the combination is unpredictable without knowledge of the underlying signaling mechanism, as described herein.

In the methods described herein the combination of the PD-1 inhibitor and the LAG-3 inhibitor preferably are administered after the initial administration of the anti-tumor vaccine. In some embodiments, “booster” or additional dosages of the anti-tumor vaccine are provided in intervals after the initial administration, e.g., 4 weeks, 6 weeks, 10 weeks, 12 weeks, 3-months, 6 months, 12 months after the initial administration.

An “effective treatment” refers to treatment producing a beneficial effect, e.g., amelioration of at least one symptom of a cancer. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing one or more symptoms of the cancer or metastasis thereof. Such effective treatment may, e.g., reduce patient pain, reduce the size or number of cancer cells, may reduce or prevent metastasis of a cancer cell, or may slow cancer or metastatic cell growth. The

The terms “cancer,” “tumor” or “malignancy” are used throughout this description interchangeably and refer to the diseases of abnormal cell growth. While the present disclosure is directed to the treatment of prostate cancer, in some embodiments, castrate-resistant prostate cancer, one of skill in the art could readily extend the present teachings to other known solid cancers using cancer specific DNA vaccines. Suitable cancers include, without limitation, breast cancer, prostate cancer, cervical cancer, ovarian cancer, pancreatic cancer, glioblastoma, melanoma, renal cell carcinoma, melanoma, colon cancer, colorectal cancer, sarcoma, kidney cancer, and those summarized in “Cancer DNA vaccines: current preclinical and clinical developments and future perspectives” Lopes et al. Journal of Experimental and Clinical Cancer Research” 38, 146 (2019), the contents of which are incorporated by reference in its entirety. Preferably, the cancer is breast cancer, cervical cancer, colorectal cancer, prostate cancer, lymphoma and sarcoma, and more preferably a cancer that is resistant to PD-1 therapy.

As used herein, “castrate-resistant prostate cancer” refers to prostate cancer that keeps growing even when the amount of testosterone in the body is reduced to very low levels. Many early-stage prostate cancers need normal levels of testosterone to grow, but castrate-resistant prostate cancers do not. Thus, castrate-resistant prostate cancer describes prostate cancer that is no longer responding to treatments that reduce androgens in the subject.

The terms “metastasis” or “secondary tumor” refer to cancer cells that have spread to a secondary site, e.g., outside of the original primary cancer site. Secondary sites include, but are not limited to, for example, the lymphatic system, skin, distant organs (e.g., liver, stomach, pancreas, brain, etc.) and the like and will differ depending on the site of the primary tumor.

Preferably, in some embodiments, the tumor is resistant to PD-1 therapy. The term “refractory” or “resistant” to checkpoint inhibitors or PD-1/PD-L1 inhibitors refers to subjects that have been treated with the checkpoint inhibitors and/or PD-1/PD-L1 inhibitors and the cancer has either developed resistance to the therapy or has responded poorly or not responded to the treatment with the inhibitors even at the beginning of treatment.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a mammalian, for example, human, subject. Preferably, the human subject has a cancer, and in some embodiments, a cancer resistant to PD-1 therapy.

For purposes of the present invention, “treating” or “treatment” describes the management and care of a subject for combating the disease, condition, or disorder. Treating includes the administration of the multicell conjugate or composition described herein to reduce, prevent, ameliorate and/or improve the onset of the symptoms or complications, alleviating the symptoms or complications, or reducing or eliminating the disease, condition, or disorder.

For example, treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation.

The present disclosure further provides a method of increasing the anti-tumor immune response, and in some embodiments, a T cell response to a tumor antigen in a subject having cancer, the method comprising administering an effective amount of a DNA vaccine, and a combination of a PD-1 inhibitor and a LAG-3 inhibitor, wherein the combination is effective in increasing the anti-tumor immune response to the tumor antigen of the vaccine as compared to the vaccine alone.

In some embodiments, the anti-tumor immune response is a cellular immune response. Preferably, the cellular immune response is a T cell response. Suitable T cell responses include, for example, a CD8+ T cell response or a cytotoxic T lymphocyte (CTL) response. Cellular immune responses are understood by one skilled in the art, and include the ability to kill tumor cells. Activation of CD8+ T cells leads to programmed cell death of the tumor cells. In some embodiments, anti-tumor immune response is measured by assessing the cytotoxicity of T cells, for example, by cytotoxicity assays known in the art, or by assessing production of effector molecules, e.g., interferon gamma (IFN-γ), tumor necrosis factor α (TNFα), etc., by T cells from the subject.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intratumoral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. The vaccine and the inhibitors described herein may be administered via different routes, for example, the vaccine may be administered by injection (e.g., intramuscular, intradermal, etc.) while the inhibitors may be administered by intravenous administration, oral administration, etc. depending of the inhibitor selected.

In another embodiment, the present disclosure provides compositions and kits for eliciting an anti-tumor response to a tumor cell. The composition or kit comprises at least one DNA vaccine to a tumor antigen; at least one PD-1 inhibitor; and at least one LAG-3 inhibitor. In some embodiments, the anti-tumor vaccine is a DNA vaccine to the tumor antigen, wherein the tumor antigen is synovial sarcoma X breakpoint 2 (SSX2), androgen receptor ligand-binding domain (AR LBD), prostate-specific antigen (PSA), human epidermal growth factor receptor 2 (HER-2/neu), or prostatic acid phosphatase (PAP).

The inhibitors and vaccines used in the methods of the present invention may be formulated in any form that is appropriate for administration to the subject. For example, one or more of the inhibitors or vaccines may be formulated with a pharmaceutically acceptable carrier. The term “pharmaceutically acceptable carrier” refers to any carrier, diluent or excipient that is compatible with the other ingredients of the formulation and not deleterious to the recipient. Preferably, the pharmaceutically acceptable carrier is chosen in accordance with the selected route of administration and standard pharmaceutical practice for each agent. For example, for oral administration, the active ingredient may be combined with one or more solid inactive ingredients for the preparation of tablets, capsules, pills, powders, granules or other suitable oral dosage forms. For instance, the active agent may be combined with excipients such as fillers, binders, humectants, disintegrating agents, solution retarders, absorption accelerators, wetting agents absorbents or lubricating agents. Alternatively, for parenteral administration, the active agent may be mixed with a suitable carrier or diluent such as water, an oil (e.g., a vegetable oil), ethanol, saline solution (e.g., phosphate buffer saline or saline), aqueous dextrose (glucose) and related sugar solutions, glycerol, or a glycol such as propylene glycol or polyethylene glycol. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

Further, inhibitors or vaccines used in the methods of the present invention may be formulated into dosage forms according to standard practices in the field of pharmaceutical preparations. See, e.g., Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, injectable solutions, troches, suppositories, or suspensions. For antibodies, suitable dosages forms are typically solutions.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The references cited herein are explicitly incorporated by reference in their entireties.

### Exemplary Embodiments

In one embodiment, a method of treating a subject having cancer is provided. The method comprises administering an anti-tumor vaccine and a combination of a PD-1 inhibitor and an LAG-3 inhibitor, wherein the combination is effective in increasing the efficacy of the anti-tumor vaccine and treating the cancer.

In another embodiment, a method of increasing the anti-tumor T cell response to a tumor antigen in a subject having cancer is provided. The method comprises administering an effective amount of a DNA vaccine and a combination of PD-1 inhibitor and an LAG-3 inhibitor, wherein the combination is effective in increasing the anti-tumor T cell immune response. In some aspects, the subject has a cancer resistant to PD-1.

The method of any one of the preceding embodiments, the subject has a cancer selected from breast cancer, cervical cancer, colorectal cancer, prostate cancer, lymphoma and sarcoma.

In some aspects, the DNA vaccine comprises a polynucleotide encoding the tumor antigen, wherein the tumor antigen is selected from the group consisting of synovial sarcoma X breakpoint 2 (SSX2), androgen receptor ligand-binding domain (AR LBD), prostate-specific antigen (PSA), human epidermal growth factor receptor 2 (HER-2/neu), and prostatic acid phosphatase (PAP). In a preferred aspect, the cancer is prostate cancer. the cancer is prostate cancer.

The method of any one of the preceding embodiments, wherein the cancer is castrate resistant prostate cancer.

The method of any one of the preceding embodiments wherein the PD-1 inhibitor is an anti-PD 1 antibody.

The method of any one of the preceding embodiments, wherein the LAG-3 inhibitor is an anti-LAG3 antibody.

The method of any one of embodiments described herein, wherein the immune response is a cellular immune response. In some aspects, the immune response is a CD8+ T cell response. In some aspects, the anti-tumor vaccine is a DNA vaccine to the tumor antigen.

The method of any one of the preceding embodiments, wherein the combination of the PD-1 inhibitor and the LAG-3 inhibitor is administered after the anti-tumor vaccine in the subject.

In another embodiment, the disclosure provides a method of increasing the immune response to a tumor antigen on a cell in a subject, the method comprising contacting the subject with at least one vaccine directed to said tumor antigen, at least one PD-1 inhibitor and at least one LAG-3 inhibitor, wherein the immune response to said tumor antigen is increased relative to a subject treated with the tumor vaccine alone. In some aspects, the immune response is a cellular immune response. In some aspects, the immune response is a CD8+ T cell response. In some aspects, the PD-1 inhibitor is an anti-PD1 antibody.

In some aspects of the methods of any one of embodiments, the LAG-3 inhibitor is an anti-LAG3 antibody. In some aspects, the tumor vaccine is a DNA vaccine. In some aspects, the tumor is a prostate cancer. In some aspects, the tumor is resistant to PD-1 inhibitor treatment alone.

In another embodiment, the disclosure provides a kit for eliciting an anti-tumor response, the kit comprising: at least one DNA vaccine to a tumor antigen; at least one PD-1 inhibitor; and at least one LAG-3 inhibitor. In some embodiments, the DNA vaccine to the tumor antigen, wherein the tumor antigen is synovial sarcoma X breakpoint 2 (SSX2), androgen receptor ligand-binding domain (AR LBD), prostate-specific antigen (PSA), human epidermal growth factor receptor 2 (HER-2/neu), or prostatic acid phosphatase (PAP).

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The invention will be more fully understood upon consideration of the following non-limiting examples.

### EXAMPLE 1

In this example, rational dual checkpoint blockade combinations and test these combinations in tumor models. The inventors used three separate murine tumor models targeting different antigens with different vaccines: C57BL/6 mice implanted with E.G7-OVA tumors expressing ovalbumin which the inventors previously modified to overexpress PD-L1 (PD-L1^{high}),19 an HLA-A2+ HLA-DR1+ (HHD-II) mouse model in which mice were implanted with sarcoma cells expressing the human synovial sarcoma X breakpoint 2 (SSX2) protein as a tumor antigen,20 and FVB mice implanted with MycCaP prostate tumor cells, using a vaccine targeting the native androgen receptor (AR).21-23 Using OT-1 mice, the inventors assessed immune checkpoint expression on CD8+T cells following activation by antigen alone or by antigen presented by professional APC. The inventors found that PD-1, CTLA-4, LAG-3, and TIM-3 were all upregulated in the presence of professional APC. However, in the absence of professional APC, LAG-3 was the only checkpoint molecule expressed, suggesting LAG-3 as a rational target for dual blockade in combination with anti-tumor vaccination. Subsequent studies focused on anti-tumor vaccination in the presence or absence of PD-1, LAG-3, or dual PD-1/LAG-3 antibody blockade. The inventors found that in a model less responsive to vaccination and PD-1 blockade anti-tumor vaccination produced a greater anti-tumor response when used in combination with both PD-1 and LAG-3 blockade.

### Materials and Methods:

### Mice

HLA-A2.01/HLA-DR1-expressing (HHDII-DR1) mice on a C57BL/6 background were obtained from Charles River Labs courtesy of Dr. François Lemonnier.24 OT-1 (Stock No: 003831), C57BL/6 J (B6, Stock No: 000664), and FVB/NJ (FVB, Stock No: 001800) mice were purchased from The Jackson Laboratory (Jax, Bar Harbor, Mass.). All mice were maintained and treated in microisolator cages under aseptic conditions, and all experiments were conducted under an IACUC-approved protocol that conforms to the NIH guide for the care and use of laboratory animals.

### Cell Lines

E.G7-OVA (derivative of EL4) cells were obtained from ATCC (Manassas, Va., Cat. #CRL-2113) and maintained via the ATCC-recommended culture methods. E.G7-OVA cells were lentivirally transduced to express PD-L1, as previously described.19 The A2/sarcoma cell line expressing SSX2 (A2/Sarc-SSX2) was generated as previously described.16 The MycCaP cell line was obtained from ATCC (Cat #CRL-3255) and cultured according to their instructions. All cell lines used were authenticated and tested for mycoplasma.

### Peptides

Peptides encoding the H2K^{b}-restricted epitope from chicken ovalbumin (SIINFEKL) and the HLA-A2 restricted epitope of SSX2 (RLQGISPKI) were synthesized, and the purity and identity confirmed by mass spectrometry and gas chromatography (LifeTein, LLC., Hillsborough, N.J.). Peptides were dissolved in DMSO (2 mg/ml) and stored at −80° C. until required.

### In Vitro Assay

Splenocyte Stimulation

Spleens were collected from OT-1 mice, processed through a mesh screen, and splenocytes were isolated by centrifugation after red blood cell osmotic lysis with ammonium chloride/potassium chloride lysis buffer (0.15 M NH4Cl, 10 mM KHCO3, 0.1 mM EDTA). Splenocytes were cultured at 2×10⁶/mL in RPMI 1640 medium supplemented with L-glutamine, 10% fetal calf serum (FCS), 200 U/mL penicillin/streptomycin, 1% sodium pyruvate, 1% HEPES, 50 μM β-MeOH, and 2 μg/mL SIINFEKL (SEQ ID NO: 7) peptide or the HLA-A2 restricted sequence from SSX2 (RLQGISPKI (SEQ ID NO: 8)) as a nonspecific control.

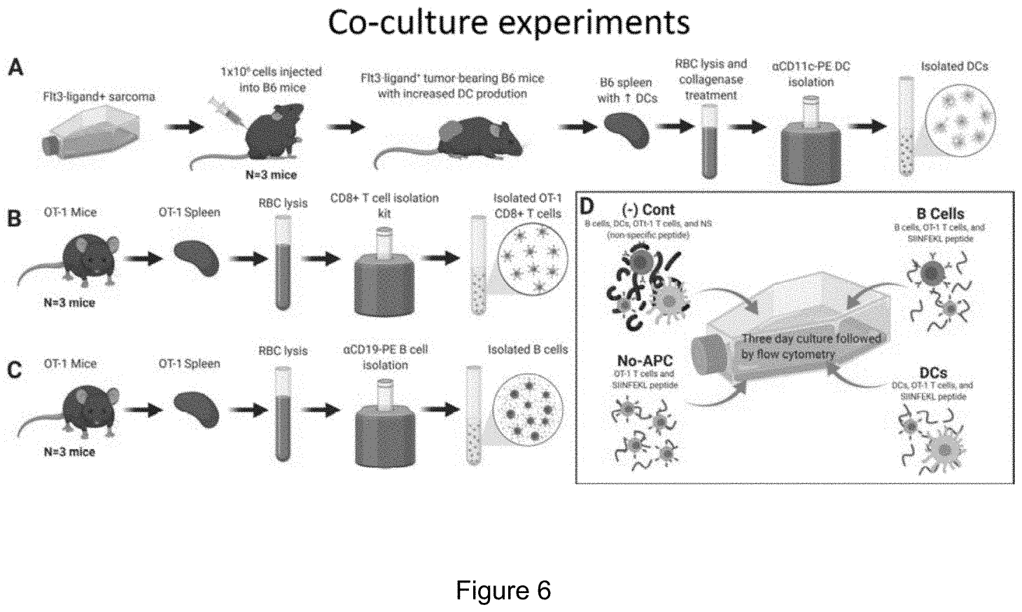

Co-Culture Experiments (FIG. 6)

B cells or dendritic cells (DCs) were enriched from splenocytes of OT-1 or B6 mice inoculated with Flt3 ligand-expressing B16 tumor cells25 using PE-labeled antibodies specific for either CD19 or CD11c (StemCell, Seattle, Wash., Cat. #17,684) as previously described.26 Similarly, CD8+T cells were isolated using a negative selection CD8+T-cell isolation kit (StemCell, Cat. #19,853). After enrichment, each APC subset, and a subset of purified T cells, were cultured as described above with 2 μg/mL SIINFELK (SEQ ID NO: 7) or the HLA-A2 sequence from SSX2 (RLQGISPKI (SEQ ID NO: 8)) as a nonspecific control peptide. Naïve OT-1 T cells were added to each cell type at a 1:1 ratio and incubated for three days, after which cells were stained and analyzed by flow cytometry with the following panel: CD3-FITC (BD 555,274), CD4-BUV395 (BD 563,790), CD8-BUV805 (BD 564,920), LAG-3-BV711 (BD 563,179), PD1-PECF594 (BD 562,523), TIM3-APC (eBioscience 17-5871-82), CTLA4-PECy7 (Tonbo 60-1522-U100), 41BB-PerCPeF710 (eBioscience 46-1371-82), and Live/Dead Ghost dye 780 (Tonbo, San Diego, Calif. 13-0865-T100).

Immunization Studies

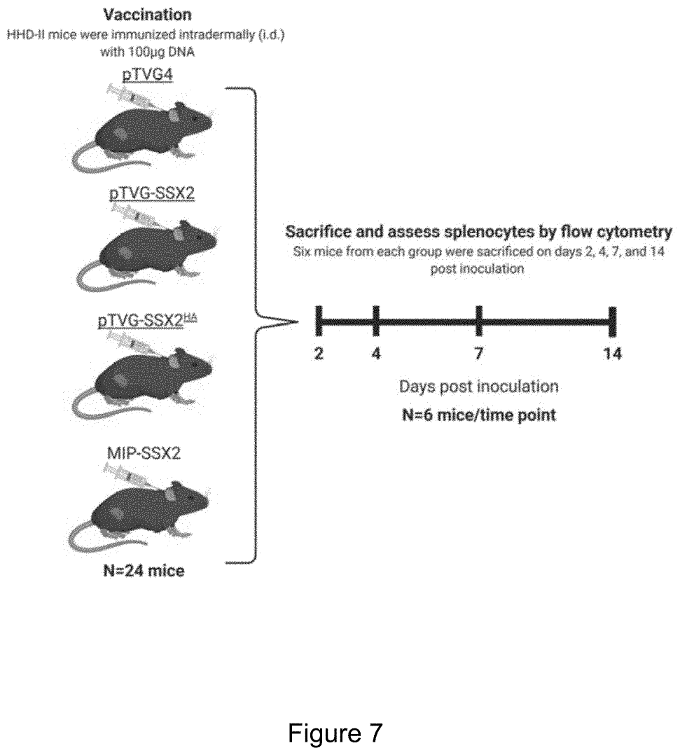

The construction of DNA vaccines encoding SSX2 was previously described.20 Six- to to eight-week-old HHDII-DR1 mice were randomized into treatment groups and immunized intradermally (i.d.) with the 100 μg pTVG4 control vector, pTVG-SSX2, pTVG-SSX2^{HA}, or MIP-SSX2 DNA vaccines (FIG. 7). At 2, 4, 7, 10, and 14 days after immunization, a group from each treatment type were euthanized, their spleens collected and SSX2-tetramer+ CD8 T cells assessed by flow cytometry directly for surface markers or stimulated with the dominant HLA-A2 restricted epitope of SSX2; p103-111, RLQGISPKI (SEQ ID NO: 8), for 16 hours (8 alone and 8 in the presence of BD GolgiStop [BD Biosciences, Cat. #554,724]) and activation and cytokine production of all CD8 T cells assessed by intracellular cytokine staining and flow cytometry using standard protocols provided by BD biosciences. A flow panel for direct analysis of surface markers was as described above, with the addition of SSX2 p103 tetramer-APC.

Tumor Treatment Studies

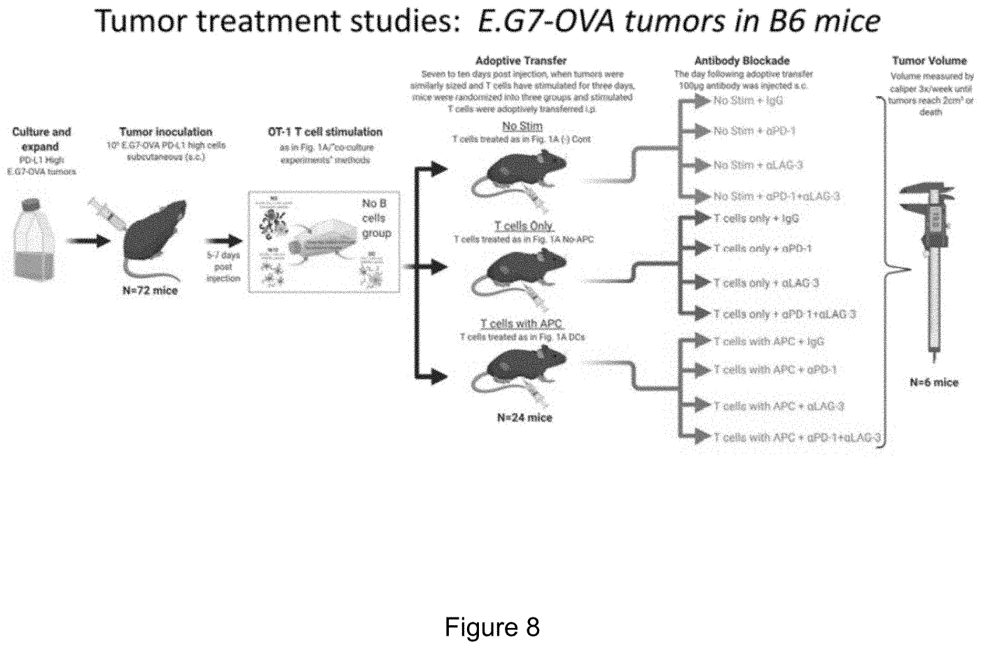

### E.G7-OVA Tumors in B6 Mice (FIG. 8)

Six- to ten-week-old female B6 mice were injected subcutaneously (s.c.) with 106 ovalbumin-expressing E.G7-OVA PD-L1high cells. Seven to ten days postinjection, when tumors were palpable and similarly sized (˜0.1 cm3), mice were randomized into treatment groups and OT-1 splenocytes were harvested and SIINFEKL-specific CD8+T cells and DC were isolated as previously described.26 OT-1 CD8+T cells were stimulated for 36 hours in the presence of 2 μg/mL SIINFEKL (SEQ ID NO: 7) or vehicle control with or without a 1:1 ratio of DC as described above. Following stimulation, three groups of T cells were isolated: those that received vehicle (No Stim), those that were simulated in the absence of DCs (No APC), and those that were stimulated in the presence of DCs (DC). Ten days after tumor implantation, 106 of each T cell subset were adoptively transferred via intraperitoneal (i.p.) injection into the E.G7-OVA PD-L1high tumor-bearing mice. The day following transfer, mice were given 100 μg αPD-1, αLAG-3, both αPD-1 and αLAG-3, or IgG control. Tumor volume was measured with calipers three times weekly until tumors reach 2 cm3 or death and calculated in cubic centimeters using the following formula: (π/6)*(long axis, cm)*(short axis, cm)2. Animals with tumors larger than 2 cm3 were compassionately euthanized.

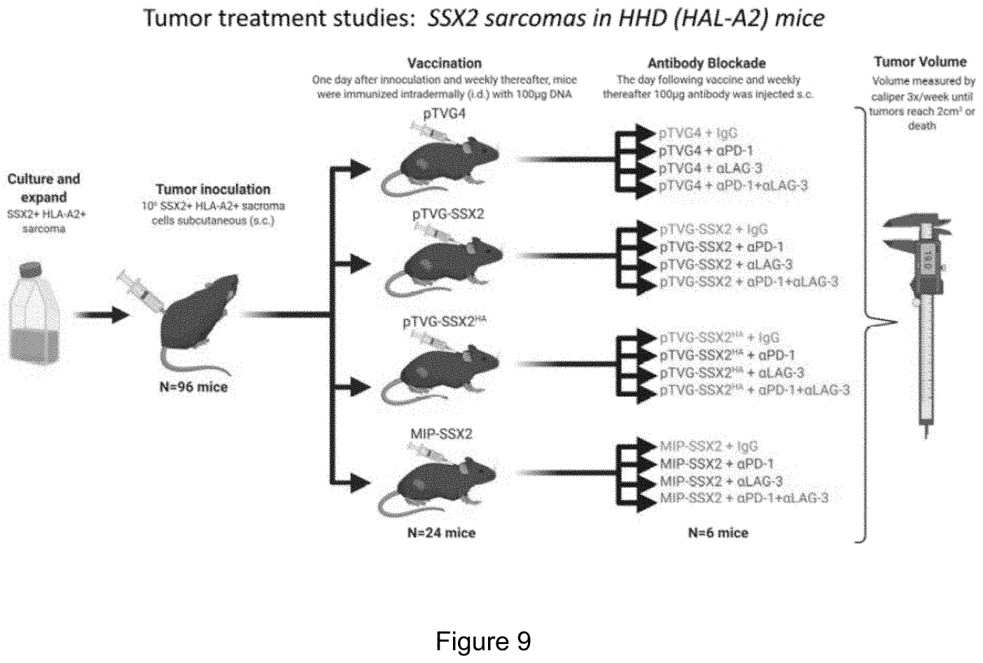

### SSX2+ Sarcomas in HHD-II Mice (FIG. 9)

Six- to eight-week-old female HHDII-DR1 mice were inoculated with 10⁵ A2/Sarc-SSX2 cells administered s.c. in 50% Matrigel (Corning, Tewksbury, Mass. Cat. #354,248). The following day, mice were immunized i.d. with 100 μg pTVG4 control vector, pTVG-SSX2, pTVG-SSX2^{HA}, or MIP-SSX2 DNA, and the day after that, each vaccine group was administered 100 μg i.p. αPD-1, αLAG-3, both αPD-1/αLAG-3, or IgG control antibodies. Tumor volume was measured over time, with endpoints as above.

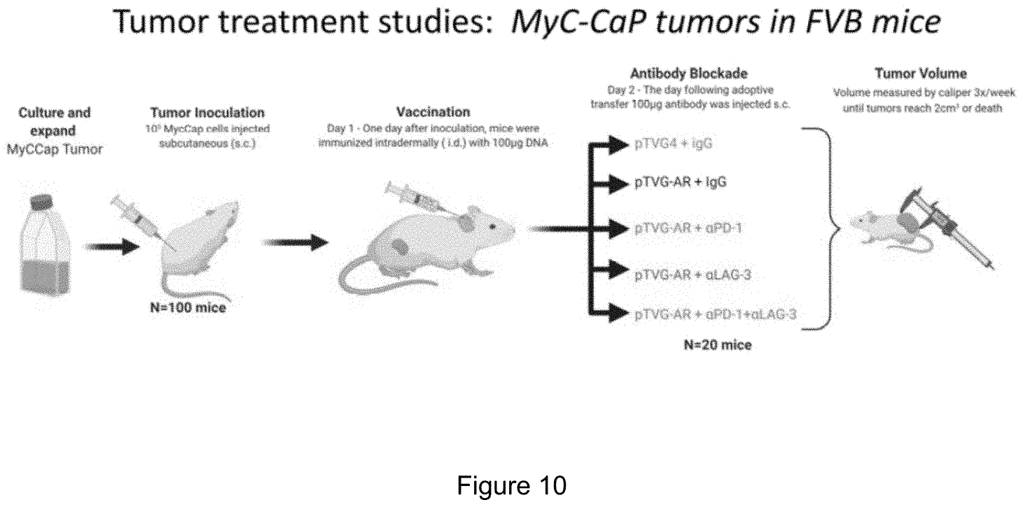

### MycCaP Tumors in FVB Mice (FIG. 10)

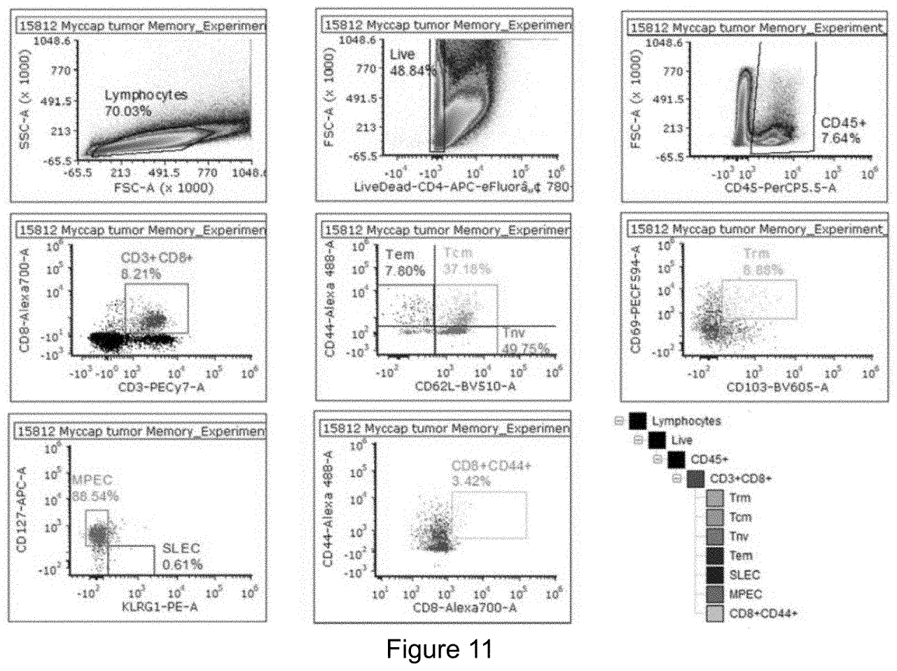

6- to 9-week-old male FVB mice were injected s.c. with 10⁶ MycCaP cells on day 0. Beginning the next day (day 1) and continuing weekly, mice were immunized i.d. with 100 ug pTVG4 control vector or pTVG-AR vaccine. The following day (day 2), and weekly thereafter, mice were injected i.p. with 100 μg of IgG, αPD-1, αLAG-3, or both αPD-1/αLAG-3 (100 μg each). Tumor volume was measured over time, with endpoints as above. In a parallel study, tumors were also collected on day 29, digested with collagenase, and assessed by flow cytometry as described above, with the gating strategy as shown in FIG. 11.

### Statistical Analyses

Comparison of group means was performed using GraphPad Prism software, v8.4.3. Analysis of Variance (ANOVA) followed by the Bonferroni multiple-comparison post-hoc procedure was used to compare individual group means. Where ANOVA was not possible, comparison of group means was performed using the mixed effects model with Geisser-Greenhouse correction. Survival analysis was conducted using a Mantel-Cox log-rank test. For all comparisons, P values equal to or less than 0.05 were considered statistically significant.

### Results

### T-Cell Activation by Professional APCs can Lead to Distinct Immune Checkpoint Expression on CD8±T Cells

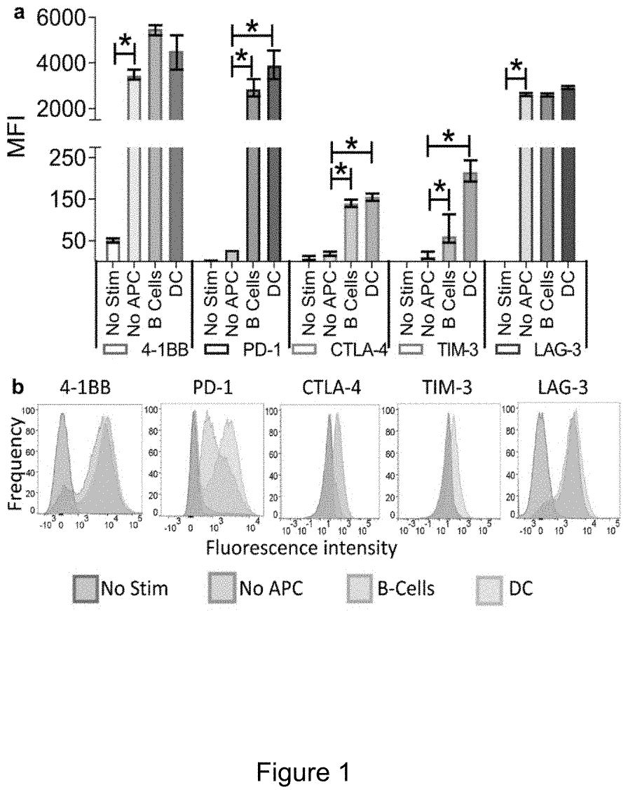

As described earlier, the inventors' previous work has demonstrated that differences in T-cell priming from anti-tumor vaccination can lead to expression of different checkpoint receptors which can impede the anti-tumor efficacy of vaccine induced CD8+T cells.16·18·19 To evaluate this further, the inventors first assessed the expression of checkpoints immediately following antigen encounter by activating OT-1 CD8+T cells with SIINFEKL peptide in the presence or absence of professional APC (DCs or B cells). Shown in FIG. 1 are the mean fluorescence intensities (MFI) of 4-1BB (CD137, as a marker of T-cell activation), PD-1, CTLA-4, TIM-3, and LAG-3 on OT-1 CD8+T cells activated in the presence or absence (No Stim) of cognate SIINFEKL (SEQ ID NO: 7) peptide. Expression of all the checkpoint receptors and 4-1BB was increased on cells stimulated in the presence of professional APCs (either B cells or DC). Expression of TIM-3 was slightly (but not significantly, p=0.086) lower when B cells were used as professional APC compared to DC. However, when T cells were stimulated alone without professional APC, the only checkpoint receptor with increased expression was LAG-3. This suggests that activation with co-stimulation leads to expression of other receptors and LAG-3 is increased with activation in the absence of a co-stimulatory signal.

### Blockade of PD-1 or LAG-3 Improves Anti-Tumor Activity of Activated CD8±T-Cells

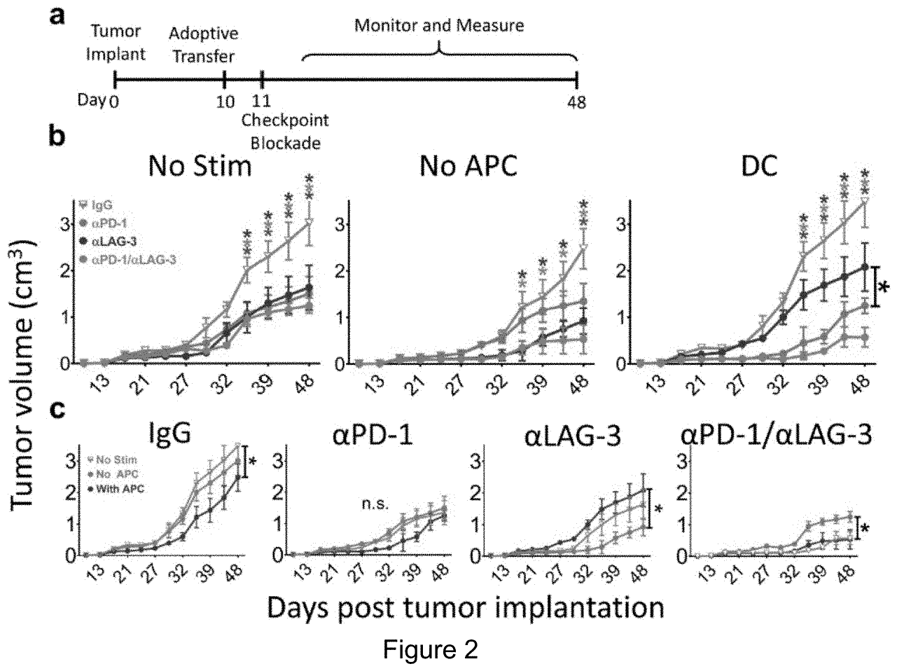

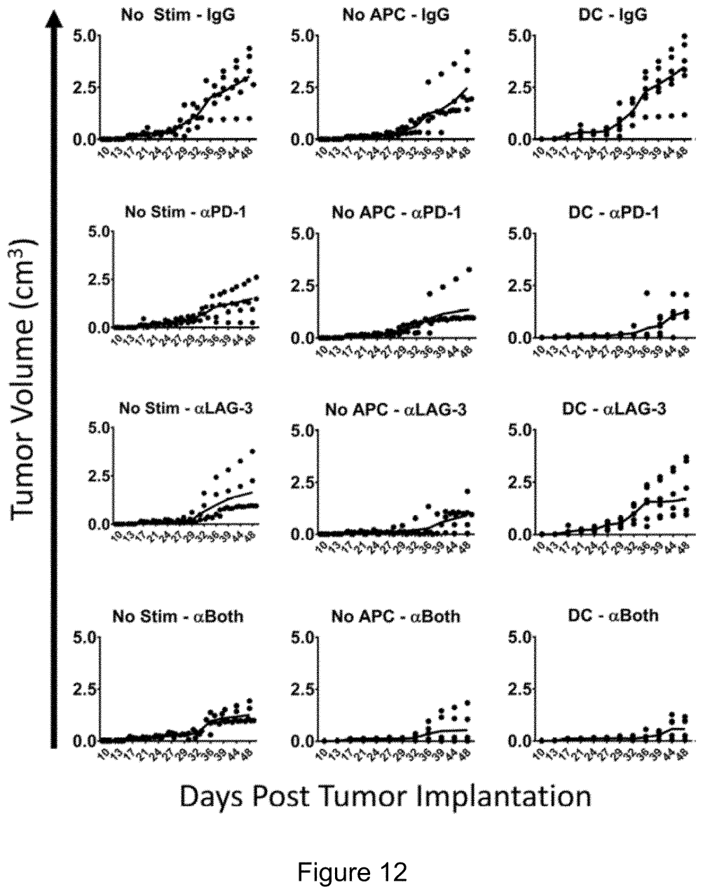

To determine directly whether expression of specific receptors interferes with anti-tumor response and whether blocking activation-induced checkpoint receptors can ameliorate the anti-tumor response, naïve OT-1+ CD8+T cells, or OT-1+ CD8+T cells that were stimulated in vitro with or without APC (DC), were adoptively transferred to B6 mice bearing PD-L1^{high} E.G7-OVA tumors. Following the transfer, mice were administered IgG isotype, αPD-1, αLAG-3, or both αPD-1 and αLAG-3 monoclonal antibodies (FIG. 2a). As shown in FIG. 2b, all groups that received checkpoint blockade had marked reductions in tumor growth when compared to IgG. However, LAG-3 blockade was most effective when used with T cells stimulated without APC (FIG. 2c). Blockade of both PD-1 and LAG-3 produced a greater delay in tumor growth when compared to IgG or LAG-3, however the response following dual blockade was not significantly greater when compared to PD-1 alone in this model (individual growth curves shown in FIG. 12).

### DNA Vaccination Can Elicit CD8±T Cells Differentially Expressing PD-1 and LAG-3

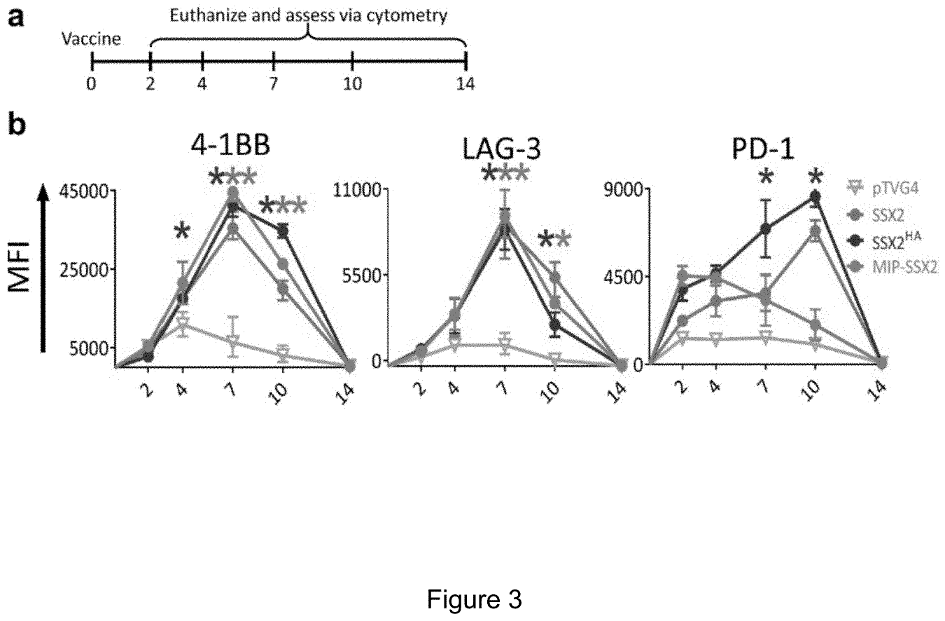

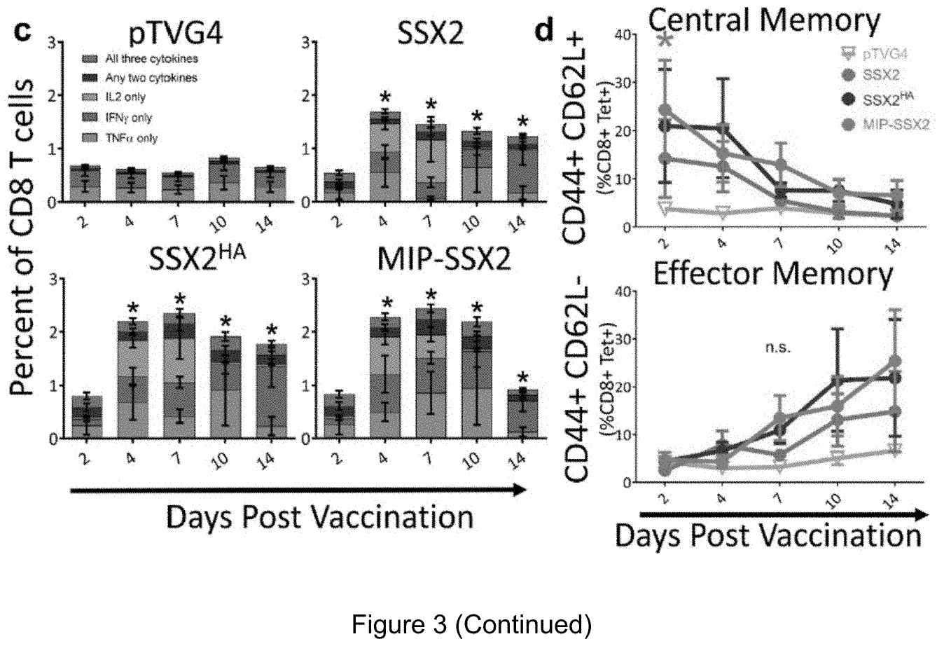

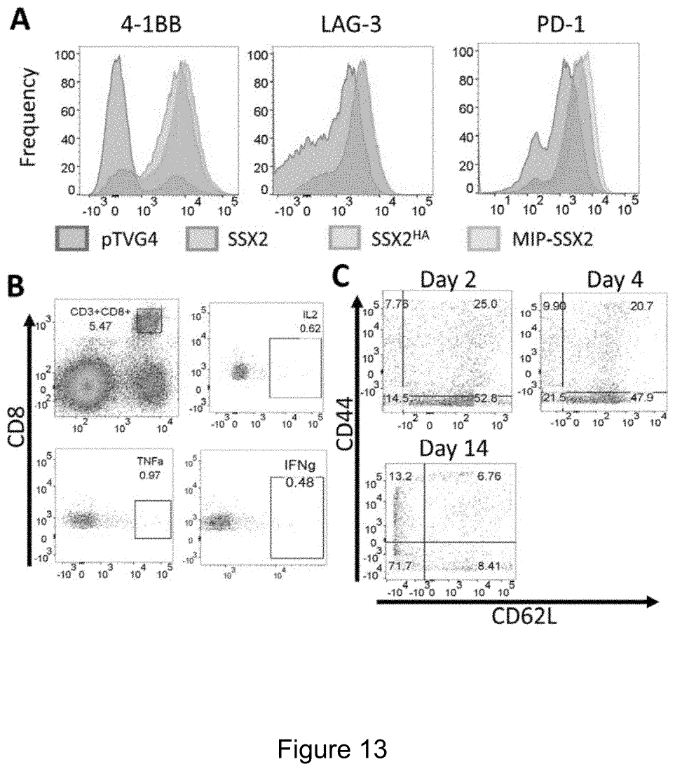

The inventors next wished to determine how PD-1 and/or LAG-3 blockade, when used concurrently with DNA vaccination, would affect the resulting Th1 CD8+T-cell response. For this, the inventors first evaluated HLA-A2/DR1+ HHD-II mice vaccinated with different plasmid vectors encoding SSX2. Specifically, pTVG-SSX2^{HA} encodes two epitopes with high HLA-A2 affinity and was previously demonstrated to elicit antigen-specific CD8+T cells with higher PD-1 expression compared to a vector encoding the native SSX2 epitopes (pTVG-SSX2).16 The other construct, mini-intronic plasmid SSX2 (MIP-SSX2), encodes the native SSX2 protein in a mini-intronic plasmid resulting in prolonged expression of SSX2 in vivo, and was previously demonstrated to elicit antigen-specific CD8+T cells with higher LAG-3 expression compared to pTVG-SSX2.18 Mice were immunized with one of these modified vaccines, the native pTVG-SS2 vaccine, or empty vector (pTVG4). Splenocytes from immunized animals were collected at 2, 4, 7, 10 and 14 days after immunization to assess checkpoint expression and memory phenotype (FIG. 3a). As shown in FIG. 3b, immunization with pTVG-SSX2^{HA} led to p103 (the dominant HLA-A2 epitope for SSX2) tetramer+ CD8+T cells with increased PD-1 expression when compared to the other vaccines (representative histograms are shown in FIG. 13). Immunization with MIP-SSX2 predominantly induced LAG-3 expression, with lower expression of PD-1 compared to the other vectors. These findings were consistent with the inventors' previous findings.16·18 As shown in FIG. 3c, vaccination with any of the SSX2 constructs elicited CD8+T cells with similar Th1 cytokine profiles following in vitro stimulation with the p103 peptide epitope. As shown in FIG. 3d, each of the SSX2 vaccines led to a similar transition from central to effector CD8 memory, which is expected following cytotoxic T-cell expansion (representative dot plots in FIG. 13).27

PD-1 Blockade is Superior to LAG-3 Blockade when Used in Combination with an Anti-Tumor Vaccine in an αPD-1 Sensitive Tumor

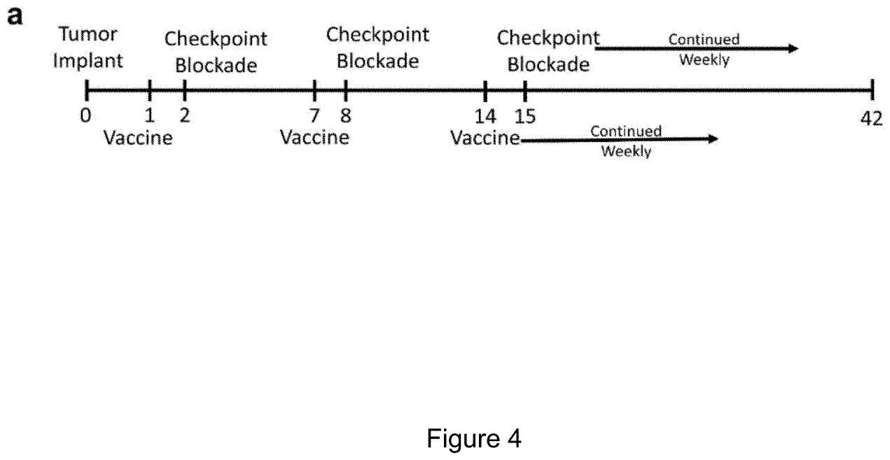

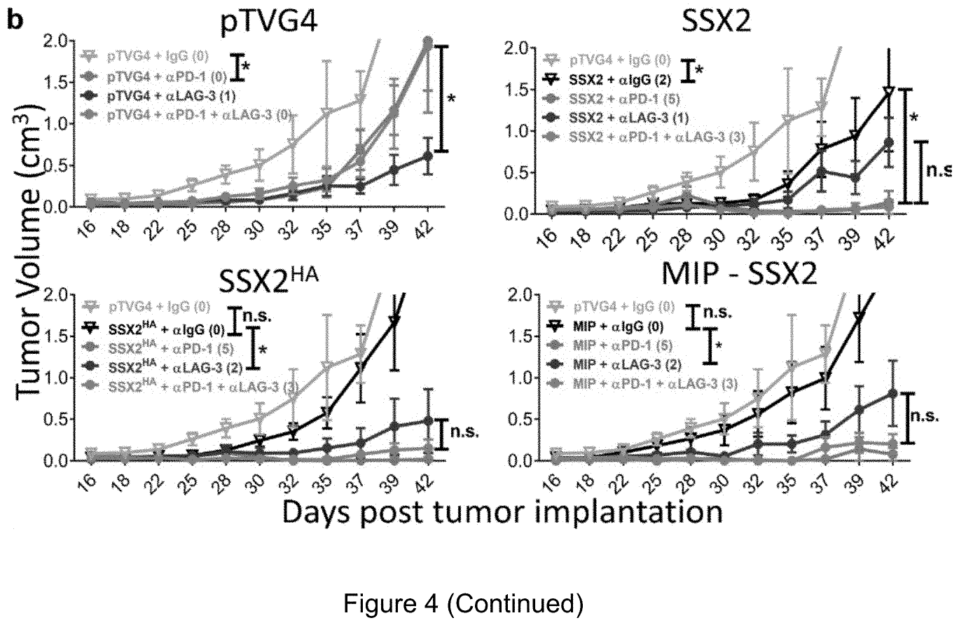

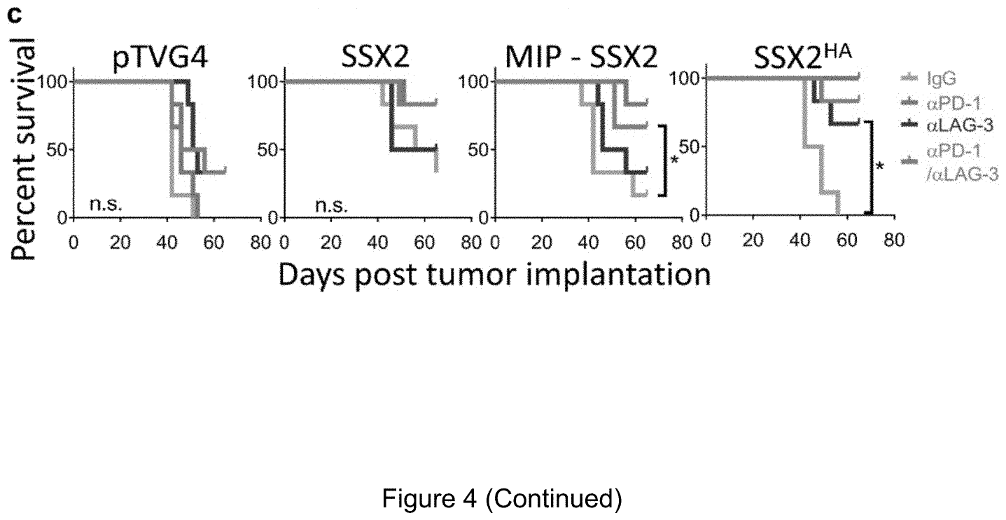

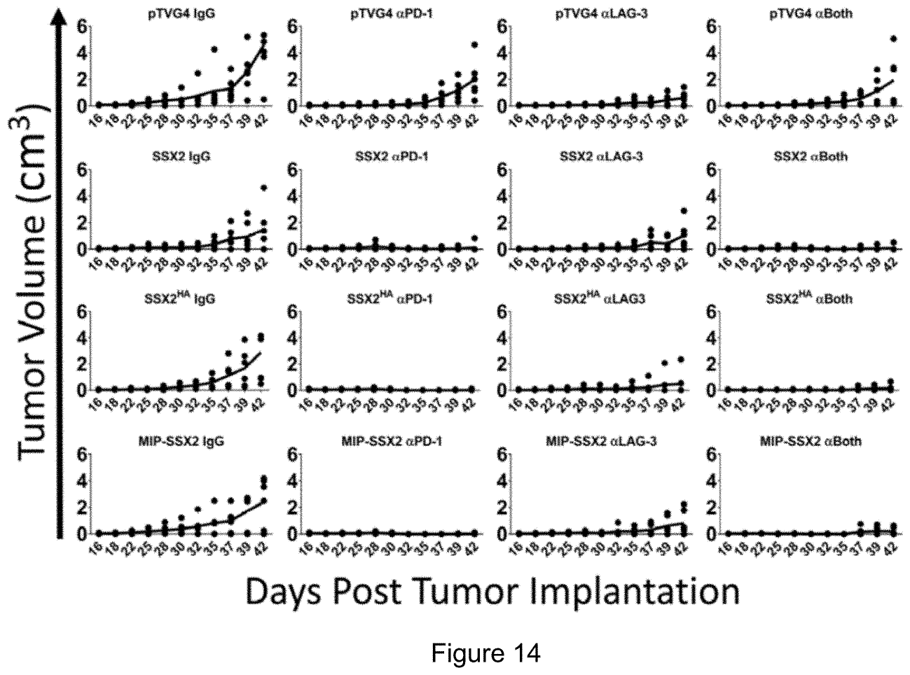

The inventors next wished to determine whether PD-1 and LAG-3 blockade was superior to either alone when used in combination with these anti-tumor DNA vaccines. 6- to 8-week-old HLA-A2+ HHD-II mice were inoculated with SSX2-expressing sarcoma cells. As shown in FIG. 4a, the mice were immunized with pTVG4 control vector, pTVG-SSX2, pTVG-SSX2^{HA}, or MIP-SSX2 DNA vaccines one day following tumor implantation and at weekly intervals thereafter. The day following each immunization, mice were administered αPD-1, αLAG-3, both αPD-1/αLAG-3, or IgG control antibodies. Shown in FIG. 4 are the mean tumor sizes (4B) and survival curves (4C) from each treatment group (individual data points are shown in FIG. 14). Consistent with the inventors' previous findings, and despite the similar cytokine expression profile and memory phenotype of CD8+T cells described in FIG. 3, pTVG-SSX2^{HA} and MIP-SSX2 vaccines were inferior to the native pTVG-SSX2 vaccine when used without T-cell checkpoint blockade (pTVG-SSX2 vs pTVG-SSX2^{HA}p=0.036; pTVG-SSX2 vs MIP-SSX2 p=0.026). However, when the altered vaccines were used in combination with checkpoint blockade, all blocking antibodies resulted in reduced tumor growth when compared to IgG control. As in the PD-L1^{high} E.G7-OVA tumors, both αPD-1 and the αPD-1/αLAG-3 combination slowed tumor growth to a greater extent and prolonged survival when compared to αLAG-3 alone with antigen-specific vaccination. However, the response to vaccination with dual αPD-1/αLAG-3 blockade was not significantly greater than blockade with αPD-1 alone (pTVG-SSX2p=0.99; pTVG-SSX2^{HA}p=0.84; MIP-SSX2p=0.92), which in this model was highly effective. A treatment response was observed with αLAG-3 and control vector in this particular experiment, but not observed in repeated studies (data not shown).

In a Prostate Cancer Model, Vaccination with PD-1 and LAG-3 Blockade is Superior to Vaccination with Either Blockade Alone

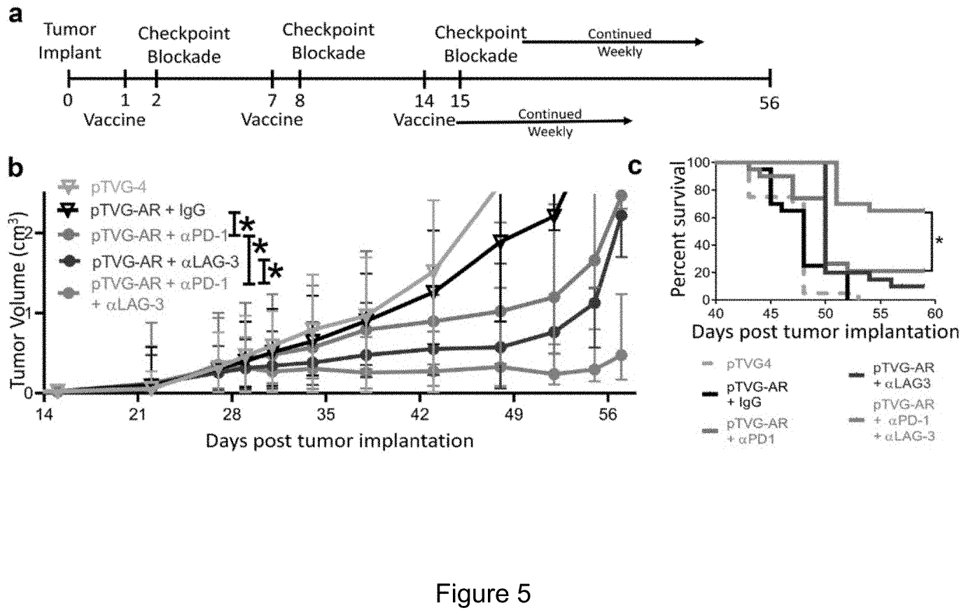

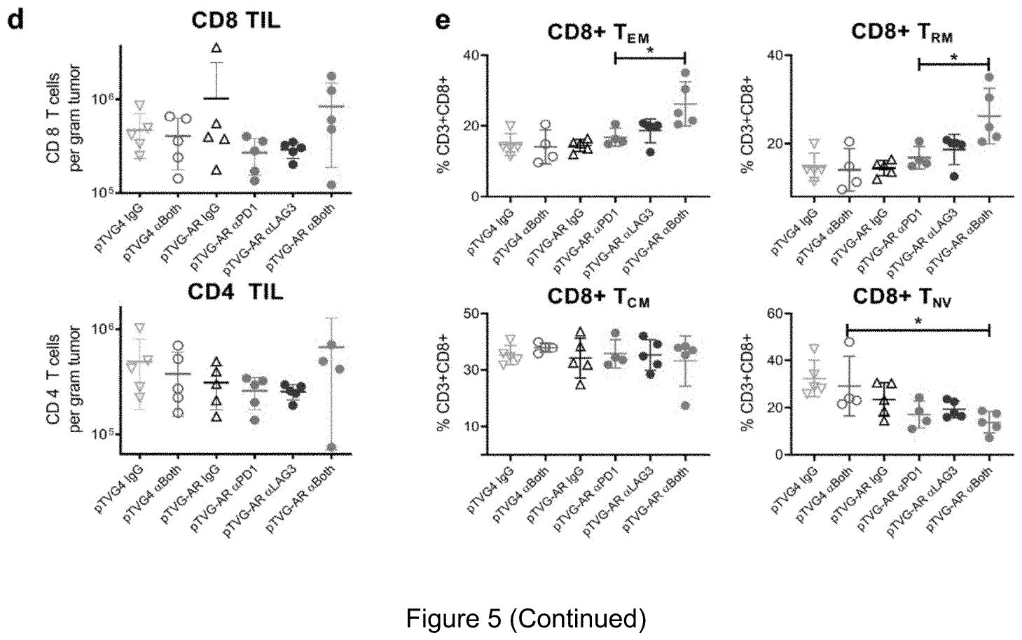

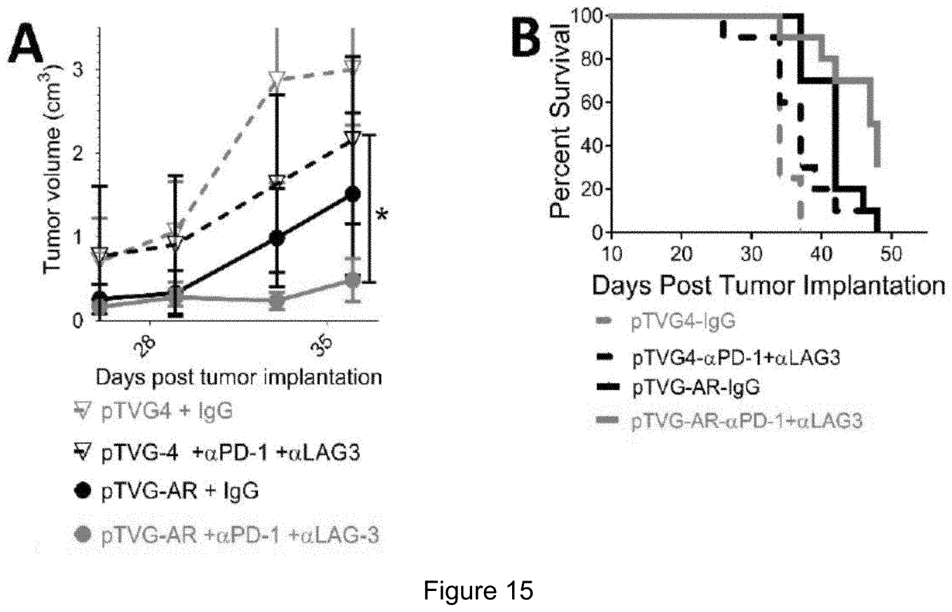

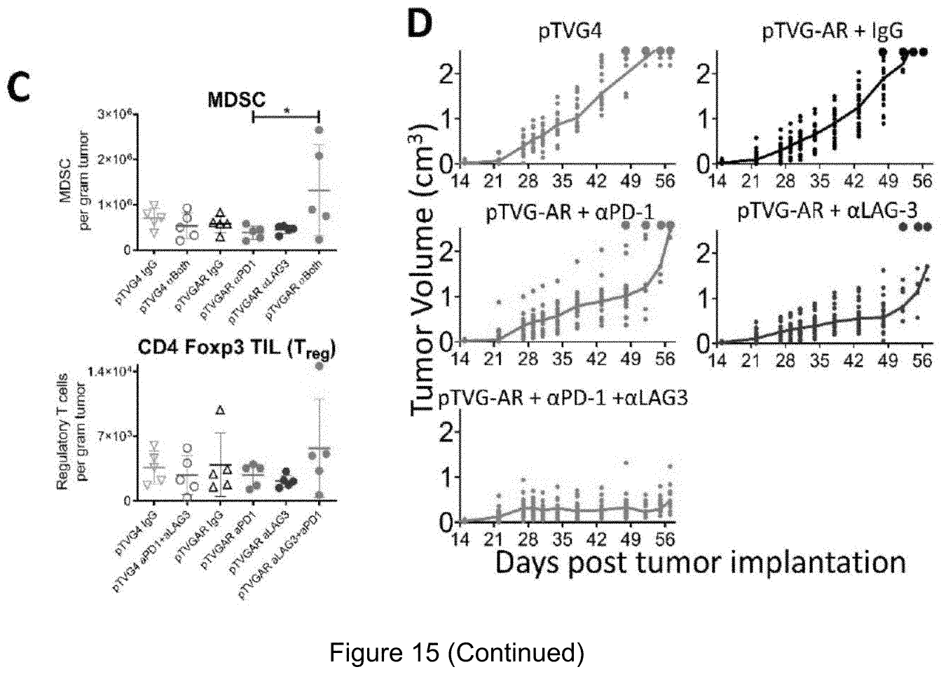

The inventors next wished to evaluate vaccination with checkpoint blockade in a murine model less responsive to PD-1 blockade. Prostate cancers have been considered mostly resistant to single-agent PD-1 blockade in clinical trials, and previous reports using the murine MycCaP prostate cancer model have demonstrated that while it does respond to anti-tumor vaccination, it does not respond to PD-1/PD-L1 blockade.23·28-30 As shown in FIG. 5a, six-to-nine-week-old male FVB mice were inoculated with MycCaP cells and immunized with the pTVG4 control or a DNA vaccine encoding the native ligand-binding domain of the androgen receptor (pTVG-AR). The day following immunization and weekly thereafter, mice were treated with αPD-1, αLAG-3, both αPD-1/αLAG-3, or IgG control. As shown in FIG. 5b and FIG. 5c, all vaccine combinations slowed the growth of tumors when compared to the vaccine with IgG; however, the combination of αPD-1 and αLAG-3 with vaccine led to a significant reduction in tumor growth compared to vaccination with either antibody alone. Treatment of mice with αPD-1 and/or αLAG-3 without vaccine showed little anti-tumor effect in this model (FIG. 15). In a duplicate study, tumors were collected at day 29 for further evaluation. The combination treatment led to a slight increase (not significant) in the number of infiltrating CD4+ and CD8+T cells (FIG. 5d), as well as an unexpected increase in tumor-infiltrating MDSC (FIG. 15). Further evaluation of tumor-infiltrating CD8+T cells showed these to be predominantly of an effector memory and tissue-resident memory phenotype (FIG. 5e).

### Discussion

In this example, the inventors investigated the activation-induced expression of immune checkpoint receptors on CD8+T cells and how that expression is affected by T-cells which had been activated by professional or “nonprofessional” APC. Based on this information, the inventors identified a rational combination of checkpoint inhibitors to use with anti-tumor vaccination. The inventors report that T cells stimulated in the absence of professional APC increased expression of LAG-3 but not PD-1, CTLA-4, or TIM3, while T cells stimulated with APC displayed an increase in all checkpoint receptors observed. The inventors thus focused on combinations of PD-1 and LAG-3 blockade in the context of anti-tumor vaccination. Using DNA vaccines that the inventors have previously demonstrated can lead to antigen-specific CD8+T cells with increased expression of PD-1 or LAG-3, the inventors found that either checkpoint blockade successfully enhanced vaccine induced anti-tumor responses with all the vaccines tested; however, the inventors found no specific advantage to vaccination with dual PD-1/LAG-3 blockade over vaccine with PD-1 blockade alone in murine models that were robustly sensitive to PD-1 blockade.19 In the prostate cancer model, which is resistant to single-agent PD-1 blockade, and using a vaccine encoding a naturally expressed tumor antigen, the dual blockade group demonstrated greater therapeutic efficacy than other treatment groups. These results indicate the following: 1) depending on which cells are presenting antigen, tumor-reactive CD8+T cells can be activated with distinct patterns of checkpoint receptor expression; 2) dual blockade of PD-1 and LAG-3 can provide significant benefit over either blockade alone in PD-1 resistant MycCaP prostate tumors; 3) the upregulation of other checkpoint receptors (e.g. TIM-3, CTLA-4, VISTA, CD160, BTLA etc.), and the persistence of some tumors despite activation of a Th1 biased T-cell response and targeted checkpoint blockade suggest that combination strategies with vaccine and other checkpoint blocking antibodies could be the focus of future investigations.

The inventors' approach was based on the finding that T-cell activation following vaccination resulted in the expression of PD-1, LAG-3, CTLA-4, and TIM-3 checkpoint receptors. Of note, the inventors did see a slight decrease in TIM-3 following stimulation with B cells as professional APC (FIG. 1), suggesting there could be differences in T cell function following stimulation by different professional APC types. However, in the absence of professional APC, activated CD8+T cells expressed only LAG-3. The inventors reasoned that combination checkpoint blockade following vaccination should consequently include LAG-3 blockade, as vaccines, and notably DNA vaccines, can result in antigen presentation through nonprofessional APC. The inventors have previously shown that, during T-cell activation, a longer contact time between the CD8+T cell and the APC (i.e. longer exposure to TCR signaling and co-stimulation), resulted in elevated PD-1 expression that persisted for months after antigen exposure.19 These data suggest the existence of a negative feedback loop in which excess TCR stimulation leads to the expression of PD-1 and other inhibitory receptors and molecules. Given the current study, LAG-3 expression appears to be dependent on TCR stimulation, but not necessarily co-stimulation. This suggests LAG-3 expression may be part of a second negative feedback loop that is regulated independently of PD-1, and consequently that the use of PD-1 and LAG-3 in a dual checkpoint blockade strategy could be advantageous following vaccination with a tumor antigen.

The inventors' data demonstrate that if CD8+T cells are activated in a way that leads to the expression LAG-3 alone; then, their anti-tumor activity is improved with LAG-3 blockade. However, following vaccination, there was little benefit to adding LAG-3 blockade to vaccine with PD-1 blockade in the OVA-expressing or SSX2 sarcoma models. It is possible that this is entirely due to the first two model systems being exquisitely sensitive to PD-1 blockade with vaccination. The inventors had specifically used E.G7-OVA tumor cells transfected to express PD-L1 as a model at least partially responsive to PD-1 blockade, compared with E.G7-OVA cells that do not express PD-L1.31 In addition, the inventors had previously demonstrated that OVA-specific CD8+T cells infiltrating these tumors following treatment had increased LAG-3 expression.31 However, use of this cell line with checkpoint blockade, and the inventors' model using SSX2 DNA vaccination with PD-1 blockade alone, resulted in eradication of tumors in many animals. Hence, demonstrating a benefit with combined blockade was challenging in these models. Notwithstanding, the inventors used the SSX2 sarcoma model specifically because the inventors' prior data demonstrated that altered vaccines could elicit CD8+T cells with preferential expression of PD-1 or LAG-3, and hence might respond differently to vaccination with checkpoint blockade. It is conceivable that in these tumor models because the antigens targeted are not normal “self” proteins expressed in the host, the majority of antigen-specific CD8+T cells were activated by professional APC and predominantly expressed PD-1. However, the inventors' data are consistent with a report that combined PD-1 and LAG-3 blockade was effective when used in combination with a viral vaccine targeting non-self antigens.32 Together, these data suggest that this combination might be more effective than vaccination with PD-1 blockade alone, particularly for tumors less responsive to PD-1 blockade.

In the inventors' tumor studies, while some tumors were eradicated, many were not. This was despite demonstrating activation of CD8+T cells, the infiltration of tumors by CD8+T cells and blocking one or more of the checkpoint inhibitory receptors. The inventors have similarly found in patients with advanced prostate cancer, treated with vaccine and PD-1 blockade, that while some had evidence of disease response, this was often not durable.33 Certainly many additional mechanisms of tumor immune evasion are present, but the observation that blocking multiple checkpoint receptors following vaccine leads to increased anti-tumor response suggests that combination blockade should be further explored, both in the clinic and in further preclinical studies. The inventors' findings in the MycCaP tumor model that CD11b+Gr−1+ MDSC were increased following treatment suggest that this could be an additional mechanism of resistance. Hence, the inventors' future studies will explore anti-tumor vaccines with other combinations of checkpoint blockade and/or therapies that reduce other immunosuppressive cells and pathways upregulated in the tumor microenvironment following anti-tumor vaccination.34

### REFERENCES

1. Tang F, Du X, Liu M, Zheng P, Liu Y. Anti-CTLA-4 antibodies in cancer immunotherapy: selective depletion of intratumoral regulatory T cells or checkpoint blockade? Cell Biosci. 2018; 8:30. doi: doi.org/10.1186/s13578-018-0229-z.
2. Jin H T, Anderson A C, Tan W G, West E E, Ha S J, Araki K, Freeman G J, Kuchroo V K, A hmed R. Cooperation of Tim-3 and PD-1 in CD8 T-cell exhaustion during chronic viral infection. Proc Natl Acad Sci USA. 2010; 107(33):14733-10. doi: doi.org/10.1073/pnas.1009731107.
3. Pardoll D M. The blockade of immune checkpoints in cancer immunotherapy. Nat Rev Cancer. 2012; 12(4):252-264. doi: doi.org/10.1038/nrc3239.
4. Riley J L. PD-1 signaling in primary T cells. Immunol Rev. 2009; 229(1):114-125. doi: doi.org/10.1111/j.1600-065X.2009.00767.x.
5. Hui E, Cheung J, Zhu J, Su X, Taylor M J, Wallweber H A, Sasmal D K, Huang J, Kim J. M, Mellman I, et al. T cell costimulatory receptor CD28 is a primary target for PD-1-mediated inhibition. Science. 2017; 355(6332):1428-1433. doi://doi.org/10.1126/science.aaf1292.
6. Hodi F S, O'Day S J, McDermott D F et al. Improved survival with ipilimumab in patients with metastatic melanoma. N Engl J Med. 2010; 363(8):711-723. doi://doi.org/10.1056/NEJMoa1003466.
7. Hamid O, Robert C, Daud A, Hodi F S, Hwu W J, Kefford R, Wolchok J D, Hersey P, Joseph R W, Weber J S, et al. Safety and tumor responses with lambrolizumab (anti-PD-1) in melanoma. N Engl J Med. 2013; 369(2):134-144. doi:doi.org/10.1056/NEJMoa1305133.
8. Sakuishi K, Apetoh L, Sullivan J M, Blazar B R, Kuchroo V K, Anderson A C. Targeting Tim-3 and PD-1 pathways to reverse T cell exhaustion and restore anti-tumor immunity. J Exp Med. 2010; 207(10):2187-2194. doi:doi.org/10.1084/jem.20100643.
9. Larkin J, Chiarion-Sileni V, Gonzalez R, Grob J J, Cowey C L, Lao C D, Schadendorf D, Dummer R, Smylie M, Rutkowski P, et al. Combined nivolumab and ipilimumab or monotherapy in untreated melanoma. N Engl J Med. 2015; 373(1):23-34. doi:doi.org/10.1056/NEJMoa1504030.
10. Crosby E J, Wei J, Yang X Y, Lei G, Wang T, Liu C X, Agarwal P, Korman A J, Morse M A, Gouin K, et al. Complimentary mechanisms of dual checkpoint blockade expand unique T-cell repertoires and activate adaptive anti-tumor immunity in triple-negative breast tumors. Oncoimmunology. 2018; 7(5):e1421891. doi:doi.org/10.1080/2162402X.2017.1421891.
11. Fiegle E, Doleschel D, Koletnik S, Rix A, Weiskirchen R, Borkham-Kamphorst E, Kiessling F, Lederle W. Dual CTLA-4 and PD-L1 blockade inhibits tumor growth and liver metastasis in a highly aggressive orthotopic mouse model of colon cancer. Neoplasia. 2019; 21(9):932-944. doi:doi.org/10.1016/j.neo.2019.07.006.
12. Shi L Z, Goswami S, Fu T, Guan B, Chen J, Xiong L, Zhang J, Ng Tang D, Zhang X, Vence L, et al. Blockade of CTLA-4 and PD-1 enhances adoptive T-cell therapy efficacy in an ICOS mediated manner. Cancer Immunol Res. 2019; 7(11):1803-1812. doi:doi.org/10.1158/2326-6066.CIR-18-0873.
13. Kos S, Lopes A, Preat V, Cemazar M, Lampreht Tratar U, Ucakar B, Vanvarenberg K, Sersa G, Vandermeulen G. Intradermal DNA vaccination combined with dual CTLA-4 and PD-1 blockade provides robust tumor immunity in murine melanoma. PLoS One. 2019; 14(5):e0217762. Doi doi.org/10.1371/journal.pone.0217762.
14. DiGiulio S (2015) FDA approves Opdivo-yervoy combo for melanoma, first combo immunotherapy regimen for cancer. FDA Actions Updates. journals.lww.com/oncology-times/blog/fdaactionsandupdates/pages/post.aspx?PostID=116. 2020
15. Motzer R J, Tannir N M, McDermott D F, Aren Frontera O, Melichar B, Choueiri T K, Plimack E R, Barthelemy P, Porta C, George S, et al. Nivolumab plus ipilimumab versus sunitinib in advanced Renal-cell carcinoma. N Engl J Med. 2018; 378(14):1277-1290. doi: doi.org/10.1056/NEJMoa1712126.
16. Rekoske B T, Smith H A, Olson B M, Maricque B B, McNeel D G. PD-1 or PD-L1 blockade restores antitumor efficacy following SSX2 Epitope-modified DNA vaccine immunization. Cancer Immunol Res. 2015; 3(8):946-955. doi: doi.org/10.1158/2326-6066.CIR-14-0206.
17. Zumwalde N A, Domae E, Mescher M F, Shimizu Y. ICAM-1-Dependent homotypic aggregates regulate CD8 T cell effector function and differentiation during T cell activation. The Journal of Immunology. 2013; 191(7):3681-3693. doi: doi.org/10.4049/jimmunol.1201954.
18. Colluru V T, Zahm C D, McNeel D G. Mini-intronic plasmid vaccination elicits tolerant LAG3(+) CD8(+) T cells and inferior antitumor responses. Oncoimmunology. 2016; 5(10):e1223002. doi: doi.org/10.1080/2162402X.2016.1223002.
19. Zahm C D, Colluru V T, McNeel D G. Vaccination with high-affinity epitopes impairs antitumor efficacy by increasing PD-1 expression on CD8(+) T cells. Cancer Immunol Res. 2017; 5(8):630-641. doi: doi.org/10.1158/2326-6066.CIR-16-0374.
20. Smith H A, McNeel D G. Vaccines targeting the cancer-testis antigen SSX-2 elicit HLA-A2 epitope-specific cytolytic T cells. J Immunother (Hagerstown, Md: 1997). 2011; 34(8):569-580. doi: doi.org/10.1097/CJI.0b013e31822b5b1d.
21. Smith H A, Rekoske B T, McNeel D G. DNA vaccines encoding altered peptide ligands for SSX2 enhance epitope-specific CD8+ T-cell immune responses. Vaccine. 2014; 32(15):1707-1715. doi: doi.org/10.1016/j.vaccine.2014.01.048.
22. Smith H A, McNeel D G. Vaccines targeting the cancer-testis antigen SSX-2 elicit HLA-A2 epitope-specific cytolytic T cells. J Immunother. 2011; 34(8):569-580. doi: doi.org/10.1097/CJI.0b013e31822b5b1d.
23. Olson B M, Bradley E S, Sawicki T, Zhong W, Ranheim E A, Bloom J E, Colluru V T, Johnson L E, Rekoske B T, Eickhoff J C, et al. Safety and immunological efficacy of a DNA vaccine encoding the androgen receptor Ligand-binding domain (AR-LBD). Prostate. 2017; 77(7):812-821. doi: doi.org/10.1002/pros.23321.
24. Pajot A, Michel M L, Fazilleau N, Pancre V, Auriault C, Ojcius D M, Lemonnier F A, Lon e Y C. A mouse model of human adaptive immune functions: HLA-A2.1-/HLA-DR1-transgenic H-2 class I-/class II-knockout mice. Eur J Immunol. 2004; 34(11):3060-3069. doi: doi.org/10.1002/eji.200425463.
25. Kapadia D, Sadikovic A, Vanloubbeeck Y, Brockstedt D, Fong L. Interplay between CD8alpha+ dendritic cells and monocytes in response to Listeria monocytogenes infection attenuates T cell responses. PloS One. 2011; 6(4):e19376. doi: doi.org/10.1371/journal.pone.0019376.
26. Colluru V T, McNeel D G. B lymphocytes as direct antigen-presenting cells for anti-tumor DNA vaccines. Oncotarget. 2016; 7(42):67901-67918. doi: doi.org/10.18632/oncotarget.12178.
27. Schlub T E, Badovinac V P, Sabel J T, Harty J T, Davenport M P. Predicting CD62L expression during the CD8(+) T-cell response in vivo. Immunol Cell Biol. 2010; 88(2):157-164. doi:https://doi.org/10.1038/icb.2009.80.
28. Philippou Y, Sjoberg H T, Murphy E, Alyacoubi S, Jones K I, Gordon-Weeks A N, Phyu S, Parkes E E, Gillies McKenna W, Lamb A D, et al. Impacts of combining anti-PD-L1 immunotherapy and radiotherapy on the tumour immune microenvironment in a murine prostate cancer model. Br J Cancer. 2020; 123(7):1089-1100. doi:https://doi.org/10.1038/s41416-020-0956-x.
29. Olson B M, Johnson L E, McNeel D G. The androgen receptor: a biologically relevant vaccine target for the treatment of prostate cancer. Cancer Immunology, Immunotherapy: CII. 2013; 62(3):585-596. doi:https://doi.org/10.1007/s00262-012-1363-9.
30. Olson B M, Gamat M, Seliski J, Sawicki T, Jeffery J, Ellis L, Drake C G, Weichert J, McN eel D G. Prostate cancer cells express more Androgen Receptor (AR) following androgen deprivation, improving recognition by AR-specific T cells. Cancer Immunol Res. 2017; 5(12):1074-1085. doi:https://doi.org/10.1158/2326-6066.CIR-16-0390.
31. Zahm C D, Colluru V T, McNeel D G. Vaccination with High-affinity epitopes impairs antitumor efficacy by increasing PD-1 expression on CD8(+) T cells. Cancer Immunol Res. 2017; 5(8):630-641. doi:https://doi.org/10.1158/2326-6066.CIR-16-0374.
32. Roy S, Coulon P G, Prakash S, Srivastava R, Geertsema R, Dhanushkodi N, Lam C, Nguyen V, Gorospe E, Nguyen A M, et al. Blockade of PD-1 and LAG-3 immune checkpoints combined with vaccination restores the function of antiviral Tissue-resident CD8(+) TRM cells and reduces ocular herpes simplex infection and disease in HLA transgenic rabbits. J Virol. 2019; 93. doi:https://doi.org/10.1128/JVI.00827-19.
33. McNeel D G, Eickhoff J C, Wargowski E, Zahm C, Staab M J, Straus J, Liu G. Concurrent, but not sequential, PD-1 blockade with a DNA vaccine elicits anti-tumor responses in patients with metastatic, castration-resistant prostate cancer. Oncotarget. 2018; 9(39):25586-25596. doi:https://doi.org/10.18632/oncotarget.25387.
34. Zahm C D, Johnson L E, McNeel D G. Increased indoleamine 2,3-dioxygenase activity and expression in prostate cancer following targeted immunotherapy. Cancer Immunol Immunother. 2019; 68(10):1661-1669. doi:https://doi.org/10.1007/s00262-019-02394-w.

1. A method of treating a subject having cancer, the method comprising administering an anti-tumor vaccine and a combination of a PD-1 inhibitor and an LAG-3 inhibitor, wherein the combination is effective in increasing the efficacy of the anti-tumor vaccine and treating the cancer.

2. The method of claims 1, wherein the subject has a cancer resistant to PD-1 inhibitor.

3. The method of claim 1, wherein the subject has a cancer selected from breast cancer, cervical cancer, colorectal cancer, prostate cancer, lymphoma and sarcoma.

4. The method of claim 1, wherein the cancer is prostate cancer.

5. The method of claim 1, wherein the cancer is castrate-resistant prostate cancer.

6. The method of claim 1, wherein the DNA vaccine comprises a polynucleotide encoding the tumor antigen, wherein the tumor antigen is selected from the group consisting of synovial sarcoma X breakpoint 2 (SSX2), androgen receptor ligand-binding domain (AR LBD), prostate-specific antigen (PSA), human epidermal growth factor receptor 2 (HER-2/neu), and prostatic acid phosphatase (PAP).

7. The method of claim 1, wherein the DNA vaccine comprises a DNA vaccine selected from the group consisting of pTVG-SSX2, pTVG-SSX2^{HA}, MIP-SSX2, and pTVG-AR.

8. The method of claim 1, wherein the PD-1 inhibitor is an anti-PD-1 monoclonal antibody and the LAG-3 inhibitor is an anti-LAG3 monoclonal antibody.

9. The method of claim 1, wherein the combination of the PD-1 inhibitor and the LAG-3 inhibitor is administered after the anti-tumor vaccine in the subject.

10. A method of increasing the anti-tumor T cell response to a tumor antigen in a subject having cancer, the method comprising administering an effective amount of a DNA vaccine and a combination of PD-1 inhibitor and an LAG-3 inhibitor, wherein the combination is effective in increasing the anti-tumor T cell immune response.

11. The method of claim 10, wherein the subject has a cancer resistant to PD-1 inhibitor

12. The method of claim 9, wherein the subject has a cancer selected from breast cancer, cervical cancer, colorectal cancer, prostate cancer, lymphoma and sarcoma.

13. The method of claim 10, wherein the cancer is prostate cancer.

14. The method of claim 10, wherein the cancer is castrate-resistant prostate cancer.

15. The method of claim 10, wherein the DNA vaccine comprises a polynucleotide encoding the tumor antigen, wherein the tumor antigen is selected from the group consisting of synovial sarcoma X breakpoint 2 (SSX2), androgen receptor ligand-binding domain (AR LBD), prostate-specific antigen (PSA), human epidermal growth factor receptor 2 (HER-2/neu), and prostatic acid phosphatase (PAP).

16. The method of claim 10, wherein the DNA vaccine comprises a DNA vaccine selected from the group consisting of pTVG-SSX2, pTVG-SSX2^{HA}, MIP-SSX2, and pTVG-AR.

17. The method of claim 10, wherein the PD-1 inhibitor is an anti-PD-1 monoclonal antibody and the LAG-3 inhibitor is an anti-LAG3 monoclonal antibody.

18. The method of claim 10, wherein the immune response is a CD8+ T cell response.

19. The method of claim 10, wherein the combination of the PD-1 inhibitor and the LAG-3 inhibitor is administered after the anti-tumor vaccine in the subject.

20. A kit for eliciting an anti-tumor response, the kit comprising:
at least one DNA vaccine to a tumor antigen;
at least one PD-1 inhibitor; and
at least one LAG-3 inhibitor.
